(19) United States
(12) Patent Application Publication
(10) Pub. No.: US 2024/0302377 A1
(43) Pub. Date: Sep. 12, 2024

(54) METHODS OF IDENTIFYING NOVEL PROTEINS AND ANTIGENS IN CANCER CELLS

(71) Applicant: Georgetown University, Washington, DC (US)

(72) Inventor: Anton Wellstein, Washington, DC (US)

(73) Assignee: Georgetown University, Washington, DC (US)

(21) Appl. No.: 18/433,891

(22) Filed: Feb. 6, 2024

Related U.S. Application Data
(63) Continuation of application No. 16/322,824, filed on Feb. 1, 2019, now Pat. No. 11,927,591.
(60) Provisional application No. 62/370,020, filed on Aug. 2, 2016.

(51) Int. Cl. G01N 33/574; C12Q 1/68; C12Q 1/6886; G01N 33/68

(52) U.S. Cl. CPC G01N 33/57492; C12Q 1/68; C12Q 1/6886; G01N 33/57484; G01N 33/6848

(57) ABSTRACT

The present invention provides methods of identifying neopeptides in abnormal cells, with the methods comprising sequencing long-read messenger RNA (mRNA) isolated from the abnormal cells, identifying splice variants that could be generated from the sequenced long-read mRNA, determining if the abnormal cells contain neopeptides that correlate with the identified splice variants. The methods may also comprise identifying at least one neoantigen on the neopeptides that are present in the abnormal cells.

### CROSS-REFERENCE TO RELATED APPLICATIONS

This application is a continuation of U.S. patent application Ser. No. 16/322,824, filed on Feb. 1, 2019, which is a National Stage entry under 35 U.S.C. § 371 of International Application No. PCT/US2017/045057, filed on Aug. 2, 2017 and published on Feb. 8, 2018 under Publication Number WO 2018/026896, which claims the benefit of U.S. Provisional Application No. 62/370,020 filed on Aug. 2, 2016; the entireties of all of which are herein incorporated by reference.

### STATEMENT REGARDING FEDERALLY SPONSORED RESEARCH OR DEVELOPMENT

This invention was made with government support under CA051008, CA071508, CA113477, and CA177466 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 6, 2024, is named Georgetown023US2 and is 130,750 bytes in size.

### FIELD OF INVENTION

The present invention provides methods of identifying neopeptides in abnormal cells, with the methods comprising sequencing long-read messenger RNA (mRNA) isolated from the abnormal cells, identifying splice variants that could be generated from the sequenced long-read mRNA, determining if the abnormal cells contain neopeptides that correlate with the identified splice variants. The methods may also comprise identifying at least one neoantigen on the neopeptides that are present in the abnormal cells.

### BACKGROUND OF THE INVENTION

Successful treatment of cancers with Immune Checkpoint Inhibitors (ICIs) has been associated with the mutational load of tumors. The biological rationale for this association between mutational load and ICI response is that neoantigens are generated by mutations in protein coding sequences that provide a steady flow of neoantigens to prime the immune system for the production of antigen-specific tumor-infiltrating lymphocytes (TILs). It is thought that mutant protein fragments will lead to altered MHC/peptide recognition and immune cell activation. Treatment of cancer with ICIs enhance TIL functionality.

Neoantigens are also relevant for an alternative, cell-based immunotherapeutic approach, i.e., Adoptive Cell Transfer (ACT). This concept of neoantigens derived from DNA mutations has led to an intense line of investigation to uncover relevant neoantigens. There has been, however, inconsistent findings and results using the current neoantigen discovery approach, which is based on DNA mutation analysis of tumor samples by exome sequencing of genomic DNA.

The concept of neoantigens derived from mutant DNA ignores an important alternative mechanism that can also generate neoantigens in cancers. Specifically, posttranscriptional editing of primary RNA may also involve pathologic alternative splicing, which will introduce new, immunogenic peptide sequences to the immune system. Indeed, malignant progression can generate alternatively spliced transcripts not present in normal tissues. In addition, DNA mutations in intronic sequences can also alter splicing of the primary transcripts due to changes in splice donor/acceptor sites, altered primary RNA transcript structure and RNA binding due to the mutated RNA sequence. The current methods of neoantigen identification involving sequencing of exonomic DNA for mutation are therefore limited as they cannot be used to detect mutations in RNA transcripts. What is needed are methods of identifying additional neoantigens or potential neoantigens that are generated at the RNA transcript level.

### SUMMARY OF THE INVENTION

The present invention provides methods of identifying neopeptides in abnormal cells, with the methods comprising sequencing long-read messenger RNA (mRNA) isolated from the abnormal cells, identifying splice variants that could be generated from the sequenced long-read mRNA, determining if the abnormal cells contain neopeptides that correlate with the identified splice variants. The methods may also comprise identifying at least one neoantigen on the neopeptides that are present in the abnormal cells.

### BRIEF DESCRIPTION OF THE DRAWINGS

FIG. 1 depicts a transcriptome analysis of human bone marrow (BM) cells. Poly(A)+RNA was isolated from Total (T) or lineage-Negative BM cell populations (N). Lineage-negative (lin-neg) cells contain the stem cell population present in the bone marrow. Full length (long read) cDNA libraries were subjected to single molecule real-time RNA-seq (SMRT; PacBio platform) or conventional short read RNA-seq of fragmented cDNAs at 20 million (T) or 100 million (N) read depth (Illumina platform). Full length RNA-seq data were processed using the ToFU software generated at PacBio. Illumina reads were first aligned and then assembled using the Tuxedo suite. The efficiency of the double selection of lineage negative cells used here was confirmed by comparison of the abundance of standard markers of differentiated cells: CD14=6:1; CD16b=25:1; CD24=109:1; CD45=11:1; CD66b=16:1 expression ratio of lin-pos to lin-neg cells.

FIGS. 2A-2E depict mRNA isoforms of the EEF1A1 gene as an example of analyses. FIG. 2A shows the reference gene model from hg19. Arrows indicate direction of transcription.

FIGS. 2B-2E show isoforms and abundances (TPM) discovered in lineage-neg and total BM population by conventional short read (short-read) RNA-seq (FIG. 2B, FIG. 2D) or by full length (long-read) RNA-seq (FIG. 2C, FIG. 2E). Abbreviations: S, short read RNA-seq; C, canonical transcript and open reading frame (ORF); C*, transcripts with canonical ORF; ID #s of the isoforms are from the identifiers generated by the sequencing method. ORFs and a novel protein isoform (arrows) that was confirmed by mass spectrometry for a unique peptide are shown in FIGS. 3A-3D.

FIGS. 3A-3D depict proteins predicted from the transcript isoforms identified for EEF1A1. FIG. 3A shows amino acid sequence alignments including the isoform identifiers. The protein predicted from the N7-24 transcript isoform (highlighted) was validated by mass spectrometry. The respective peptide identified after tryptic digest is shown with Y86 and V345 from the canonical protein flanking the novel junction site. FIG. 3A discloses SEQ ID NOS 1-14, 14, 14-17, 14, 14, 14, 14, 14, 14, 14, 18-19, 19-38, and 117, respectively, in order of appearance. FIG. 3B and FIG. 3C show protein structure models that were generated using the Phyre2 software. The predicted structure of the canonical EEF1A1 protein P68104 (FIG. 3B) and of the new protein N7 (FIG. 3C) are shown. For the canonical protein, the template c1g7ca covered 96% of the amino acids with >90% confidence. For the novel N7 protein the c1g7ca template covered 90% of the amino acids with >90% confidence. FIG. 3D shows higher magnification of ten transcript isoform identifiers that code for the canonical protein. Abbreviations: C, canonical, refseq derived transcripts. C*, previously unknown transcript isoforms that code for the canonical protein. Transcript isoforms SN2-3 and N21-3 predict the same protein.

FIGS. 4A-4E depict mRNA isoforms of the ANXA1 gene. FIG. 4A shows the reference gene model from hg19. Arrows indicate direction of transcription. FIGS. 4B-4E show results for lineage-neg and total BM from short read RNA-seq (FIG. 4B, FIG. 4D) or full length (long-read) RNA-seq (FIG. 4C, FIG. 4E). Isoforms and abundances (TPM) discovered in lineage-neg and total BM population by short read (FIG. 4B, FIG. 4D) or full length RNA-seq (FIG. 4C, FIG. 4E). Abbreviations: S, short read RNA-seq; C, canonical transcript and open reading frame (ORF); C*, transcript with canonical ORF; ID #s of the isoforms are from the identifiers generated by the sequencing methods. ORFs are shown in FIGS. 6A-6C).

FIGS. 5A-5C depict transcript isoforms from genomic loci of different complexity and redundancies. The number of canonical exons was obtained from the hg19 gene annotation. FIG. 5A shows representative genes with 3 to 13 canonical exons and the number of respective transcript isoforms detected. FIG. 5B shows the comparison of the mean of the top five transcript isoforms for genes with 2 to 16 exons from the analysis of all bone marrow cell populations (p=0.0014; Chi-sq. for trend short read (SR) RNA-seq vs full length (FL) RNA-seq). FIG. 5C shows the transcripts and isoforms identified only by full length (long-read) RNA-seq.

FIGS. 6A-6C depict the amino acid sequence alignment and proteins predicted from the transcript isoforms identified for ANXA1 6A shows the amino acid sequence alignments including the isoform identifiers. The canonical protein and conserved repeat domains r1-r4 are highlighted. ORFs coding for the same protein are also shown. Note: Solid lines connecting protein fragments indicate contiguous amino acid sequences predicted from the respective transcript isoform. FIG. 6A discloses SEQ ID NOS 39-52, 52-53, 53, 53-54, 54-58, 54, 54, 54, 53, 53, 53, 59, 46, 45, 60, 44, 61-62, 62-65, and 65, respectively, in order of appearance. FIG. 6B shows the predicted protein structure of the canonical ANXA1 protein P04083 with repeat domains r1 to r4 indicated. FIG. 6C shows the common predicted proteins for groups of transcript isoforms, ORFs 2, 3, 12, 13, 14, 23, 26 and the canonical ORF are listed and shown with the respective color from panel 6A. Novel ORFs are indicated.

FIG. 7 depicts multiple amino acid sequence alignment predicted from transcript isoforms for ELANE and CFD. The identifiers of the transcript isoforms are included. FIG. 7 discloses SEQ ID NOS 66, 66-70, and 70-75, respectively, in order of appearance.

FIG. 8 depicts multiple amino acid sequence alignment and mass spectrometry detected peptides for HLA-A, -B and -C transcripts. Sequences predicted from transcript isoforms of HLA-A, HLA-B and HLA-C are shown, in which SEQ ID NOS 76-80, 80-82, 80, 83-102, 99, 103, 99, 104-116, and 118-121 are disclosed, respectively, in order of appearance. The identifiers of the transcript isoforms are included. Canonical amino acid sequences are highlighted. Peptide fragments are identified by mass spectrometry analysis of tryptic fragments of proteins extracted from lin-neg bone marrow cells.

### DETAILED DESCRIPTION OF THE INVENTION

The present invention provides methods of identifying neopeptide and/or neoantigens in abnormal cells, with the methods comprising long-read sequencing of messenger RNA (mRNA) isolated from the abnormal cells, identifying splice variants that could be generated from the sequenced long-read mRNA, determining if the abnormal cells contain neopeptides that correlate with the identified splice variants. The methods may further comprise identifying at least one neoantigen on the neopeptides that are present in the abnormal cells.

As used herein, the term “neoantigen” is an antigen that is formed by peptides that are normally absent from the proteome of a cell. The term antigen is used herein as it is in art and means a molecule or portion thereof that induces the production of antibodies in an organism capable of antibody production. Not all antigens can elicit an immune response, thus the term “antigenic” is not synonymous with “immunogenic.” Likewise, the term “antigen” is not synonymous with “immunogen.” As used herein, the neoantigens that are discovered using the methods of the present invention may or may not be immunogenic. In one embodiment, the neoantigens discovered using the methods of the present invention are immunogenic. In another embodiment, the neoantigens discovered using the methods of the present invention are not immunogenic to one host, e.g., a human host, but can be used to generate antibodies in other hosts to target them therapeutically.

Typically, a “neoantigen” is used in art to indicate a new antigen that is produced when a genetic mutation occurs in the cell's DNA, thus forming a different protein when the mutant DNA is transcribed and subsequently translated. As used herein, the terms protein, peptide and polypeptide are interchangeable. The present invention, however, focuses on identifying neoantigens that are formed when an abnormal splice variant occurs to generate a protein that is normally not found in the cell's proteome. Thus, the term “neoantigen” as used herein does not imply or require the presence of a mutation in the cell's genome. Nonetheless, the methods of the present invention may also be used to identify neoantigens that result from a genetic mutation in the cell's DNA, provided, however, that the neoantigens are caused by an abnormal splice event. By way of a non-limiting example, if a genetic mutation in the cell's DNA generates an abnormal splice junction, and thus leading to an abnormal splice variant, the term “neoantigen” as used herein would apply and the methods of the present invention could be used to identify the neoantigen caused by the genetic mutation.

The neoantigens of the present invention can be specific for each individual population of cells. For example, a population of cells obtained from one subject may contain neoantigens that are different from neoantigens contained in a population of cells obtained from a different subject. Thus, while the cells' DNA may be identical or nearly identical between two cell populations taken from different subjects, the neoantigens contained in the cell populations could be different. Accordingly, the present invention can be applied to methods of personalized medicine.

Typically, the methods of the present invention can be applied to any cell or population of cells. In one embodiment, the cell or population of cells that are used in the methods of the present invention are abnormal cells or are suspected of being abnormal. As used herein, the term “abnormal cells” are cells that exhibit abnormal metabolism, growth patterns, protein expression patterns or morphology. For example, abnormal cells may be cells that do not undergo apoptosis at the appropriate time or they divide more than normal. By way of another example, abnormal cells may be cells that do not appear to be morphologically normal to a clinician, such as a pathologist or histologist. In one embodiment, the cells that are subjected to the methods of the present invention are known to be abnormal cells. In another embodiment, the cells that are subjected to the methods of the present invention are suspected of being abnormal prior to identifying the neoantigens. The methods of the present invention thus can be used to confirm that a population of cells is abnormal by detecting the presence of one or more neoantigens.

The methods of the present invention are generally performed on a population of cells. The population of cells need not be a pure population of cells. For example, the cells can be obtained from a biopsy and need not be separated and/or purified prior to obtaining the RNA from the cells. In another embodiment, the population of cells is a pure or substantially pure population of cells. For example, the cells may be obtained from a tissue sample and the tissue may be digested and the cells separated and placed in culture to expand the cell population prior to subjecting the cells to the methods of the present invention. The cells can be obtained from any animal that is capable of generating antibodies, such as but not limited to mammals and birds. In specific embodiments, the cells used in the present invention are cells obtained from a mouse, rat, rabbit, ferret, cat, dog, camel, horse, cow, human or non-human primate. Generally speaking, the cells upon which the methods of the present invention are performed are split into multiple subsets to ensure that the methods of the present invention can be completed on the population of cells. To that end, the subsets into which the cells are split need not be identical to one another, both in terms of number and purity. For example, if a biopsy is obtained from a patient, the tissue fragment may be split into multiple pieces and each piece could represent a “subset” of the population of cells without purifying the cells within each portion of the biopsy. In another example, cells may be obtained from a subject and expanded in culture, and the cultured cells may be split into multiple subsets of cells using routine passaging techniques.

In select embodiments, the population of abnormal cells is considered to be a population of cancer cells. As used herein, the term “cancer cell” refers to a cell or cells that are obtained from abnormal tissues or conditions such as, but not limited to, hypertrophy, neoplasia, hyperplasia, benign and malignant tumors. As used herein, the term “tumor” is a general term that includes hypertrophies, neoplasias, hyperplasias, benign cancers and malignant cancers. Accordingly, certain embodiments of the present invention are performed on cells isolated from a hypertrophy, a neoplasia, a hyperplasia, a benign or a malignant cancer in a subject. Other types of cancer cells include abnormal cells obtained from blood-born cancers (or non-solid tumors), such as lymphomas, leukemias and the like.

The cancer cells can be from any animal, including but not limited to any mammal, such as mouse, rat, canine, feline, bovine, equine, porcine, non-human and human primates. Mammalian cells particularly suitable for cultivation in the present media include cancer cells of human origin. In addition, transformed cells or established cell lines cancer cell lines can also be used. In one embodiment, the cells are primary or secondary cancer cells. In another embodiment, the cells are not primary cells, such as cells from an established cell line, transformed cells, thawed cells from a previously frozen collection and the like. Animal cells for culturing by the present invention may be obtained commercially, for example from ATCC (Rockville, Md.), Cell Systems, Inc. (Kirkland, Wash.), Clonetics Corporation (San Diego, Calif.), BioWhittaker (Walkersville, Md.) or Cascade Biologicals (Portland, Oreg.).

In other select embodiments, the cells are primary cancer cells of an abnormal tissue obtained from a subject, with the subset of cells being portions of the abnormal tissue. As used herein, primary cancer cells are cells that have been taken directly from living tissue, such as a biopsy, and have not been passaged or only passaged one time. Thus, primary cells have been freshly isolated, often through tissue digestion and plated. Provided the cells have been passaged one time or less, primary cells may or may not be frozen and then thawed at a later time. In addition, the tissue from which the primary cancer cells are isolated may or may not have been frozen or preserved in some other manner immediately prior to processing. By “cell culture” or “culture” is meant the maintenance of the cells in an artificial, in vitro environment. The term “cell culture” also encompasses cultivating individual cells and tissues.

As can be appreciated, obtaining RNA from cells will necessarily result in their destruction, thus the population of cells may be divided into two or more subsets of cells prior to subjecting the cells to the methods of the present invention. The subsets of cells need not be identical in size. Methods of isolating mRNA from cells are well known in the art, and the invention is not limited by the manner in which the mRNA is obtained from the cells. RNA quality can, but need not, be assessed prior to analysis by standard methods known to those skilled in the arts including capillary electrophoreses.

Generally, speaking the RNA that is obtained from the cells is cytosolic RNA. In one embodiment, the RNA that is obtained from the cells and sequenced is messenger RNA (mRNA). The mRNA may or may not be poly-adenylated (poly-A) as typically occurs when mRNA is properly processed after transcription.

As used herein, the term “long range RNA,” or “long-read RNA” including “long range RNA” or “long-read mRNA,” means that the RNA that is subject to sequencing is 70 nucleotides (nts) or longer in length. Ordinarily, RNA is sequenced in “short-read” batches of less than about 70 nts and then the sequence of the RNA is deduced by piecing together these short-read sequences in order using the known genome as a template. Recent evidence suggests, however, that such methods of short-read sequencing of RNAs, do not accurately provide a full picture of the sequence and structure of the long-read RNA as it occurs in the cell. The methods of the present invention therefor require long-read sequencing of RNA molecules that are about 70 nts or longer. In select embodiments, the long-read RNA molecules that are sequenced are between about 70 nts and about 150 nts in length, between about 150 nts and about 300 nts in length, between about 300 nts and about 500 nts, between about 500 nts and about 750 nts, between about 750 nts and about 1000 nts, between about 1000 nts and about 1250 nts, between about 1250 nts and about 1500 nts, between about 1500 nts and about 1750 nts, between about 1750 nts and about 2000 nts, between about 2000 nts and about 2250 nts, between about 2250 nts and about 2500 nts, between about 2500 nts and about 2750 nts, between about 2750 nts and about 3000 nts, between about 3000 nts and about 3250 nts, between about 3250 nts and about 3500 nts, between about 3500 nts and about 4000 nts, between about 4000 nts and about 4500 nts, between about 4500 nts and about 5000 nts, between about 5000 nts and about 5500 nts, between about 5500 nts and about 6000 nts, between about 6000 nts and about 6500 nts, between about 6500 nts and about 7000 nts, between about 7000 nts and about 7500 nts, between about 7500 nts and about 8000 nts, between about 8000 nts and about 8500 nts, between about 8500 nts and about 9000 nts, between about 9000 nts and about 9500 nts or between about 9500 nts and about 10000 nts. To obtain these long-read RNA molecules, the RNA is generally not fragmented during isolation and purification. The term “fragmented,” with respect to RNA or DNA molecules is well understood in the art and means that the longer molecules are split into shorter pieces, sometimes with the use of enzymes. In select embodiments, the long-read RNA that is sequenced is not fragmented prior to sequencing.

Once isolated, the long-read RNA is sequenced. Protocols for sequencing long-read RNA molecules are well-known in the art and are disclosed in publications such as Tilgner, H. et al., Proc. Nat'l Acad. Sci., USA, 111(27):9869-9874 (2014), Tseng, E. and Underwood, J., J. Biomol. Techniques., 24 Supplement: S45 (2013), Sharon, D., et al., Nature Biotech. 31(10):1009-1014 (2013), Pan. Q., et al., Nature Genetics, 40:1413-1415 (2008), Steijger, T., et al., Nature Methods, 10:1177-1184 (2013) and U.S. Pat. Nos. 8,192,961, 8,501,405 and 8,940,507, all of which are incorporated by reference. The invention is not limited to specific methods of sequencing long-read RNAs, provided that the RNAs that are sequenced are at least about 150 nts or more.

Once sequenced, the long-read RNA is then analyzed to determine potential splice variants that may occur in the long-read RNA. The term “splice variant” is well-understood and it used as it is in the art. In short, a splice variant is a “species” of a primary RNA, which is a direct copy of the cell's DNA, that the cell processes to exclude or include exons in the final mRNA that is transported from the nucleus to the cytosol and is then translated into a peptide. Accordingly, in one embodiment, the RNA that is obtained and sequenced during the methods of the present invention is cytosolic RNA, that is not tRNA or rRNA. In another embodiment, the RNA that is obtained and sequenced during the methods of the present invention is not nuclear RNA. A single primary RNA can thus be processed to provide multiple distinct mRNA molecules, based upon the inclusion or exclusion of specific exons from the primary RNA molecule. Each separate splice variant therefore correlates to a specific peptide, based on the amino acid sequence for that the processed mRNA codes. Because each specific splice variant results in a specific peptide, identifying potential splice variants can be used to determine if a potential peptide is present in the cell's proteome. The potential splice variants that are identified with the methods of the present invention may or may not be known splice variants of the peptide.

In one embodiment, the splice variant analysis of the long-read RNAs that are sequenced during the methods of the present invention are previously unknown splice variants, i.e., a novel splice variant, of the target RNA. Methods for determining if a splice variant is novel are well-known. In particular, one of skill in the art is well-aware of internet-based databases and collections of splice variants that are readily searchable. Examples of splice variant databases on the internet include but are not limited to Protein Information Resource (PIR) (pir.georgetown.edu), ExPASy (www.expasy.org), EVDB (projects.insilico.us/SpliceMiner/), ECgene (genome.ewha.ac.kr/ECgene/), SpliceNest (splicenest.molgen.mpg.de/) and MAASE (maase.genomics.purdue.edu/), to name a few. The identified long read RNA sequence is matched to the genomic DNA sequences to determine if an interruption of the contiguous sequences could represent a splice event in which a portion of the primary RNA transcript is removed by polymerase reading of the genomic DNA. Such matching and review of the transcript to the genomic sequence is routine in the art and can be accomplished using blast search engines, which are publically available.

Once the splice variants are determined, a protein with a specific amino acid sequence can be then predicted from each of these splice variants. Each of these proteins, if novel, represents a potential set of novel neoantigens that may be present in or on the protein. As used herein, the term “neopeptide” means a peptide that is normally absent from the proteome of a cell. The term “neopeptide” as used herein does not imply or require the presence of a mutation in the cell's genome to produce the neopeptide. Nonetheless, the methods of the present invention may also be used to identify neopeptides that result from a genetic mutation in the cell's DNA, provided, however, that the neopeptides are caused by an abnormal splice event. By way of a non-limiting example, if a genetic mutation in the cell's DNA generates an abnormal splice junction, and thus leading to an abnormal splice variant, the term “neopeptide” as used herein would apply and the methods of the present invention could be used to identify the neopeptide caused by the genetic mutation.

The neopeptides of the present invention can be specific for each individual population of cells. For example, a population of cells obtained from one subject may contain neopeptides that are different from neopeptides contained in a population of cells obtained from a different subject. Thus, while the cells' DNA may be identical or nearly identical between two cell populations taken from different subjects, the neopeptides contained in the cell populations could be different.

Methods of determining the presence or absence of peptides that correspond to the identified splice variants are then carried out on the cells. Protein is extracted from the cells using standard protein extraction techniques. Obviously, the cells from which the proteins are extracted are not the identical cells from which the RNA is extracted for sequencing, thus protein is extracted from a second subset of cells, whereas RNA was extracted from a first subset of cells.

The identification of peptides that correspond to splice variants is routine in the art and the methods of the present invention are not limited by the manner in which the peptides are identified. For example, once the neopeptide is identified in the protein extract from the population of abnormal cells, mass spectrometry can then be employed on protein extracts obtained from normal cells of the same tissue origin. If the neopeptide is not present in the population of normal cells, the presence of the neopeptide can then be considered as a marker for abnormal cells and the neoantigen can, optionally, be further identified. In other words, the methods of the present invention may also include additional steps to determining in a population of normal cells the absence of the neopeptides that correlate with the identified splice variants. In other embodiments, performing additional assays on normal cells may not be necessary if it is already established in the art that a specific cell type does of or does not normally contain a specific protein within its proteome. In this instance, simply comparing the results of the proteomic analysis described herein with well-established standards will suffice for purposes of comparing proteomes in abnormal and normal populations of cells. If the presence of the neopeptide is detected in the population of abnormal cells and the absence of the neopeptide is confirmed in the normal cells, these results would indicate that the neopeptide is a marker for abnormal cells. In an additional embodiment, the confirmed neopeptide can then be used to generate an agent, e.g., an antibody or fragment thereof, that specifically binds to the neopeptide found in abnormal cells this identifying the neoantigen in the abnormal cells.

In one aspect of the present invention, determining the presence of the neopeptides comprises the use of mass spectrometry. Methods of using mass spectrometry to determine the presence of a specific protein in a sample are well known and have been reviewed in Aebersold, R. and Mann, M., Nature, 422:198-207 (2003), which is incorporated by reference.

Other methods of determining if neopeptides are present in a sample include chemically synthesizing the neopeptides and generating antibodies towards the neopeptides using standard immunological techniques to identify one or more neoantigens that are contained on the neopeptide. Once the antibodies are generated, they are applied to the protein extract obtained from the cells and the presence or absence of binding of the antibodies to the neopeptides, thus identifying a neoantigen, can then be assessed. Detecting the presence or absence of antibody binding can be achieved using routine binding assays, such as peptide arrays, ELISA, Western Blot, immunohistochemical assays and the like. One of skill in the art would be well-versed in methods of detecting antibody binding. In one aspect of the present invention, determining the presence of the neopeptides comprises generating antibodies capable of specifically binding the neopeptides and determining if the antibodies bind to the neopeptides.

As is readily appreciated, antigens can be formed not only through a linear chain of amino acids, but also through the three dimensional configuration of the peptide that it assumes in solution. Thus, any identified neopeptides can provide an array of neoantigens in a number of ways.

As used herein, the phrase “identifying a neoantigen” is not intended require the identification of a specific linear sequence of amino acids or a specific three dimensional arrangements of the neopeptides that create the neoantigen. Instead, “identifying a neoantigen” can mean generating an agent, such as an antibody or an antibody fragment, that specifically binds to the neopeptide. In other words, production of antibodies, either polyclonal or monoclonal, is included in the phrase “identifying a neoantigen.”

Any of the antibodies can be, for example, polyclonal, monoclonal, bi-specific, multispecific, human or chimeric antibodies. The antibody molecules of the invention can be of any type, e.g., IgG, IgE, IgM, IgD, IgA and IgY, class, e.g., IgG1, IgG2, IgG3, IgG4, IgA1 and IgA2 or subclass of immunoglobulin molecule. In one embodiment, the antibody that is generated to bind to the neoantigen comprises, or alternatively consists of, a polypeptide having an amino acid sequence of a VH domain, at least one VH CDR, a VL domain, or at least one VL CDR.

The antibodies or antibody fragment that is generated to bind to the neoantigen may be monovalent, bivalent, trivalent or multivalent. For example, monovalent scFvs can be multimerized either chemically or by association with another protein or substance. An scFv that is fused to a hexahistidine tag or a Flag tag can be multimerized using Ni-NTA agarose (Qiagen) or using anti-Flag antibodies (Stratagene, Inc.).

The antibodies that are generated to bind to the neoantigens may be monospecific, bispecific, trispecific or of greater multispecificity. Multispecific antibodies may be specific for different epitopes of the neopeptide or they may be specific for both the neopeptide and a heterologous epitope, such as a heterologous polypeptide or solid support material. See, e.g., PCT publications WO 93/17715; WO 92/08802; WO 91/00360; WO 92/05793; Tutt, et al, J. Immunol. 147:60 69 (1991); U.S. Pat. Nos. 4,474,893; 4,714,681; 4,925,648; 5,573,920; 5,601,819; Kostelny et al, J. Immunol. 148:1547-1553 (1992), which are incorporated by reference.

As used herein, an antibody fragment is a fragment of an antibody capable of specifically binding the same epitope that the intact antibody would bind. Examples of antibody fragments include but are not limited to Fab and F(ab′)2 fragments, Fd fragments, disulfide-linked Fvs (sdFvs), antiidiotypic (anti-Id) antibodies, including but not limited to anti-Id antibodies to antibodies that is generated to bind to the neoantigen, and epitope-binding fragments of any of the above. Fab and F(ab′)2 fragments lack the Fc fragment of intact antibody and generally clear more rapidly from the circulation, and may have less non-specific tissue binding than that of an intact antibody (Wahl et al., J. Nucl. Med. 24:316-325 (1983)). Other types of antibody fragments include but are not limited to single chain Fv fragments (scFv) that are well-known in the art. Techniques described for the production of single chain antibodies (U.S. Pat. No. 4,946,778) can be adapted to produce single chain antibodies to specific neopeptides.

The antibodies or fragments of the present invention may be prepared by any of a variety of methods. For example, cells expressing the neopeptide can be administered to an animal to induce the production of sera containing polyclonal antibodies. In one method, a preparation of neopeptides is prepared and purified to render it substantially free of natural contaminants. Such a preparation is then introduced into an animal in order to produce polyclonal antisera of greater specific activity.

Polyclonal antibodies that are generated to bind to the neoantigens may be produced according to standard techniques by immunizing a suitable animal, e.g., rabbit, goat, etc., with an antigen comprising an antigenic portion of the neopeptide. Collecting immune serum from the animal and separating the polyclonal antibodies from the immune serum can be carried out in accordance with known procedures, and screening and isolating a polyclonal antibody specific for neopeptide can be carried out with well-known procedures and as described below. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990, which is incorporated by reference.

The neoantigen may be the full length neopeptide or a portion thereof. In some embodiments the neoantigen is a peptide of from 7 to 20 amino acids in length, in particular from about 8 to 17 amino acids in length. In some embodiments, the neoantigen desirably will comprise about 3 to 8 amino acids. Neopeptides suitable for producing antibodies that are generated to bind to the neoantigens may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czemik, Methods In Enzymology, 201: 264-283 (1991); Merrifield, J. Am. Chem. Soc. 85: 21-49 (1962), which are incorporated by reference.

In some embodiments the neopeptide is administered with an adjuvant. Suitable adjuvants will be well known to those of skill in the art. Exemplary adjuvants include complete or incomplete Freund's adjuvant, RIBI (muramyl dipeptides) or ISCOM (immunostimulating complexes).

When the above-described methods are used for producing polyclonal antibodies, following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, to monitor the success of immunization, the antibody levels with respect to the antigen in serum can be monitored using standard techniques such as ELISA, RIA and the like.

In one aspect of the present invention, the antibodies or fragments thereof that are generated to bind to the neoantigens on the neopeptides are monoclonal antibodies. Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., (1981) pp. 563-681). In general, such procedures involve immunizing an animal (for example a mouse) with a neopeptide. Suitable cells can be recognized by their capacity to bind anti-neopeptide antibody. Such cells may be cultured in any suitable tissue culture medium; however, it is desirable to culture cells in Earl's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is may be desirable to employ the parent myeloma cell line (SP₂O), available from the American Type Culture Collection, Rockville, Md. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the neoantigen on the neopeptide. The secreted antibody may be recovered from tissue culture supernatant by conventional methods such as precipitation, ion exchange or affinity chromatography, or the like. Other methods of generating monoclonal antibodies include but are not limited to the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Alternatively, additional antibodies capable of binding to the neoantigens may be produced in a two-step procedure through the use of anti-idiotypic antibodies. Such a method makes use of the fact that antibodies are themselves antigens, thus it is possible to obtain an antibody which binds to a second antibody. In accordance with this method, neopeptide specific antibodies are used to immunize an animal, for example a mouse. The splenocytes of such an animal are then used to produce hybridoma cells, and the hybridoma cells are screened to identify clones which produce an antibody whose ability to bind to neopeptide-specific antibody can be blocked by a neopeptide antigen, respectively. Such antibodies comprise anti-idiotypic antibodies to neopeptide-specific antibody and can be used to immunize an animal to induce formation of neopeptide-specific antibodies.

The invention also encompasses antibody-producing cells and cell lines, such as hybridomas, as described above.

Polyclonal or monoclonal antibodies may also be obtained through in vitro immunization. For example, phage display techniques can be used to provide libraries containing a repertoire of antibodies with varying affinities for a particular antigen. Techniques for the identification of high affinity human antibodies from such libraries are described by Griffiths et al., EMBO J., 13:3245-3260 (1994); which is incorporated by reference.

The antibodies may be produced recombinantly using methods well known in the art for example, according to the methods disclosed in U.S. Pat. No. 4,349,893 or U.S. Pat. No. 4,816,567, which are incorporated by reference. The antibodies may also be chemically constructed by specific antibodies made according to the method disclosed in U.S. Pat. No. 4,676,980, which is incorporated by reference.

Once a desired neopeptide antibody is identified, polynucleotides encoding the antibody, such as heavy, light chains or both (or single chains in the case of a single chain antibody) or portions thereof such as those encoding the variable region, may be cloned and isolated from antibody-producing cells using means that are well known in the art. For example, the neoantigen binding site of the monoclonal antibody can be cloned by PCR and single-chain antibodies produced as phage-displayed recombinant antibodies or soluble antibodies in E. coli. See, e.g., Antibody Engineering Protocols, Humana Press, Sudhir Paul, Ed. (1995), which is incorporated by reference.

In another embodiment, identifying a neoantigen can also mean using the neopeptide to assay samples, e.g., serum, from a subject for the presence of binding agents, e.g, antibodies to that bind to the neopeptide. The presence of a binding agent that binds the neopeptide would indicate that the subject's immune system has recognized the neopeptide.

The neopeptides can also be used to monitor the presence or absence of binding agents, which bind to the neopeptide, in a subject. For example, the neopeptides can be used to assay samples from a subject taken at multiple time points. When there is a decrease the amount of binding agents that bind to the neopeptides over time, this decrease could be used as an indication that the number of abnormal cells is decreasing over time. Thus in one embodiment, the invention provides methods of monitoring the progression of an abnormal condition, with the methods comprising assessing in the subject the levels of the identified neopeptide or the agent that binds the neopeptide at more than one time point. For example, some embodiments of the methods of the present invention will comprise assessing in the subject the levels of the identified neopeptide or the agent that binds the neopeptide at two, three, four, five, six, seven, eight, nine, 10 or even more time points over a period of time, such as a year, two years, three, years, four years, five years, six years, seven years, eight years, nine years or even 10 years or longer.

In other words, the present invention also includes methods of monitoring the efficacy of treatment of an abnormal condition by assessing in the subject the levels of the identified neopeptide or the agent that binds the neopeptide over the course of the treatment and after the treatment. In specific embodiments, the methods of monitoring the efficacy of treatment comprise assessing in the subject the levels of the identified neopeptide or the agent that binds the neopeptide at at least one, two, three, four, five, six, seven, eight, nine or 10 or more different time points prior to the receipt of treatment for the abnormal condition and subsequently assessing in the subject the levels of the identified neopeptide or the agent that binds the neopeptide at at least one, two, three, four, five, six, seven, eight, nine or 10 or more different time points after beginning of treatment for the abnormal condition, and determining the changes, if any, in the subject the levels of the identified neopeptide or the agent that binds the neopeptide. The levels can be monitored, with, for example, the normalization or decline in the values of the profile over time being indicative that the treatment may showing efficacy.

In another embodiment, identifying a neoantigen can mean generating a nucleic acid molecule that encodes an antibody or fragment thereof that binds a neoantigen. Methods of determining a coding sequence of a protein, including an antibody, are well known in the art. In another aspect, identifying the specific linear antigenic sequence or specific antigenic configuration of the peptide is also included in the phrase “identifying a neoantigen.”

The methods of present invention may optionally include administration of the agent used to identify the neoantigen (“identifying agent”) to a population of cells suspected of being abnormal, such as cancer cells, as well as administering the identifying agent to a group of normal cells. If binding of the identifying agent is detected in the population of abnormal cells and binding of the identifying agent is not detected in the normal cells, this analysis can be used to confirm that the identified neoantigen can be used as a marker for abnormal cells.

The present invention also provides methods of identifying abnormal tissue in a subject. For example, once the neoantigens or neopeptides have been identified, the identifying agents can then be used to identify the presence or absence of the neoantigens in other samples. These “other samples” may or may not be from the same subject from which the original population of cells was obtained. Accordingly, in one embodiment, the methods of identifying abnormal tissue comprise the additional steps of administering a compound that specifically binds to the identified neoantigen and detecting the presence or absence of the binding of the component. In another embodiment, the methods of identifying abnormal tissue comprise determining the presence of the neopeptide, e.g., using mass spectroscopy, in a population of cells suspected of being abnormal.

### EXAMPLES

### Example 1—Methods of Determining Novel Splice Variants in Bone Marrow Mononuclear Cells

Freshly harvested bone marrow tissues were collected from discarded healthy human bone marrow collection filters that had been de-identified. Mononuclear cells were isolated by Ficoll gradient centrifugation. To select for lineage-negative cells, bone marrow mononuclear cells were incubated with an antibody cocktail containing antibodies against CD2, CD3, CD11b, CD11C, CD14, CD16, CD19, CD24, CD5, CD61, CD66b, and Glycophorin A (Stemcell Technologies). Lineage-positive cells bound to the antibodies were removed by magnetic beads and lineage-negative cells obtained from the flow-through. To increase purity, lineage-negative cells were enriched two times.

Sequencing of fragmented (short-read) cDNAs. Briefly, the integrity and purity of total RNA isolated from the cells were assessed using Agilent Bioanalyzer by OD260/280 ratio. 5 μg of total RNA was subjected to rRNA depletion using the RiboZero Human/Mouse/Rat kit (Epicentre Biotechnologies). cDNA was generated from the depleted RNA using random hexamers or custom primers and Superscript III (Life Technologies, Carlsbad, CA USA, catalog #18080093). The resulting cDNA was purified and fragmented using a Covaris fragmentation kit (Covaris, Inc.), profiled using an Agilent Bioanalyzer, and Illumina libraries were prepared using NEBNext reagents (New England Biolabs). The quality, quantity and the size distribution of the Illumina libraries were determined using an Agilent Bioanalyzer 2100. The libraries were then submitted for Illumina HiSeq2000 sequencing. Paired-end 90 or 100 nucleotide reads were generated and checked for data quality using FASTQC (Babraham Institute), and DNAnexus (DNAnexus, Inc) was used on the platform provided by Center for Biotechnology and Computational Biology (University of Maryland) 17. 159,043,023 non-strand specific paired-end reads were collected for the total bone marrow sample (deep sequencing). 35,126,712 strand-specific paired-end reads were collected from the lineage-negative cell sample.

Sequencing of full length (long-read) cDNAs. The integrity and purity of total RNA were assessed using Agilent Bioanalyzer and OD260/280 prior to submission. Full length cDNA synthesis was done from polyA RNA using Clontech SMARTer PCR cDNA synthesis kit (Clontech Laboratories; 23). Libraries were prepared after size selection of cDNAs into bins that contain 1-2 kb, 2-3 kb and >3 kb cDNAs by the BluePippin size selection protocol. These fractions were converted to SMRTbell libraries. Full length cDNA libraries were produced from 1 ng of poly-A RNA. Non-fragmented long read RNA-seq followed. Total RNA was submitted to Pacific Biosciences (Menlo Park, CA) or Icahn School of Medicine at Mount Sinai (New York, NY). 17 SMRT cells (7 cells 1-2 kb, 5 cells 2-3 kb, 5 cells 3-6 kb) were used to sequence the total bone marrow cell population. 12 SMRT cells were used to sequence the lineage-negative population (5 cells 1-2 kb, 5 cells 2-3 kb, 2 cells 3-6 kb).

Peptide Analysis by Nano LC-MS/MS. Proteins were extracted using 0.1% Rapigest (Waters) in 25 mM ammonium bicarbonate extracted proteins were reduced with 5 mM DTT for 60 min at 60° C. and alkylated with 15 mM iodoacetamide for 30 min in the dark. Trypsin (Promega) digestion (2.5 ng/μL) was carried out at 37° C. in Barocycler NEP2320 (PressureBioSciences) for 1 h at 37° C. and then vacuum dried in Speed-vac (Labconco). Tryptic peptides were analyzed on a NanoAcquity UPLC (Waters) by RP chromatography on a Symmetry C18 (3 μm, 180 μm, 20 mm) trap column and UPLC capillary column (BEH 300 Å, 1.7 μm, 150 mm×0.75 μm) (Waters) interfaced with 5600 TripleTOF (AB Sciex). Separation was achieved by a 250 min gradient elution with ACN containing 0.1% formic acid. The chromatographic method was composed of 5 min trapping step using 2% ACN, 0.1% formic acid at 15 μL/min and chromatographic separation at 0.4 μL/min as follows: starting conditions 2% ACN, 0.1% formic acid; 1-180 min, 2-60% ACN, 0.1% formic acid; 180-200 min, 60-95% ACN, 0.1% formic acid; 200-220 min 95% ACN, 0.1% formic acid followed by equilibration 2% ACN, 0.1% formic acid for an additional 30 min. For all runs, 5 μL of sample were injected directly after enzymatic digestion analysis used an Information Dependent Acquisition (IDA) work flow with one full scan (400-1500 m/z) and 50 MS/MS fragmentations of major multiply charged precursor ions with rolling collision energy. Mass spectra were recorded in the MS range of 400-1500 m/z and MS/MS spectra in the range of 100-1800 m/z with resolution of 30,000 and mass accuracy up to 2 ppm using the following experimental parameters: declustering potential, 80 V; curtain gas, 15; ion spray voltage, 2300 V; ion source gas 1, 20; interface heater, 180° C.; entrance potential, 10 V; collision exit potential, 11 V; exclusion time, 5 s; collision energy was set automatically according to m/z of the precursor (rolling collision energy). Data were processed using ProteinPilot 4.0 software (AB Sciex). For targeted measurement an inclusion parent mass list was created according to in-silico tryptic digest of interesting sequences.

Transcriptome alignment and assembly from Illumina data. Illumina reads were trimmed using Trimmomatic (www.usadellab.org/cms/?page=trimmomatic) with the default parameters and the reads then aligned and assembled according to the Tuxedo suite protocol as described in Trapnell, C. et al., Nat Protoc. 7, 562-578 (2012), which is incorporated by reference. The genome of reference used was GRCh37 (hg19). The genes.gtf from this reference was used to guide the read alignment during the Tophat 2 step and Cufflinks 2. Bowtie 2 indices were used for the genome reference. All computation was performed using Amazon Web Services and through the use of Starcluster software (star.mit.edu/cluster/) to manage the boxes. A Sun Grid Engine was employed to run the tasks. Reads were trimmed by Trimmomatic with the default parameters.

The conversion of ToFU abundance to TPMs was generated according to Li, B., et. al., Bioinformatics 26, 493-500 (2010), which is incorporated by reference.

Open Read Frame (ORF) predictions were generated using the ANGEL software package (github.com/PacificBiosciences/ANGEL), which publically available on github, and through the use of SerialCloner 2.6.1. ORFs accepted were the first ORFs and not necessarily the largest. In some cases both the first and the largest ORF were included and designated with letters a, b, etc., appended to the end of the name assigned.

Multiple Sequence Alignment was performed using Clustal Omega through the website (www.ebi.ac.uk/Tools/msa/clustalo). Sequence alignment was edited using BioEdit version 7.2.5. This Sequence Alignment Editor written for the windows environment was run on OS-X Yosemite on a Mac Book Pro through the use of the wine version 1.6.2, a windows emulator available for download and installation through Home Brew version 0.9.5. For figures herein, a simplification of the names generated by the cDNA Primer software was used creating a per figure unique nomenclature relating to the consensus deposited gene structures as well as using the uniprot deposited protein isoforms to unify results.

The full length sequence reads were aligned to hg19 reference genome using gsnap. The fragmented sequence reads were aligned to hg19 reference using Tophat 2 and the aligned and paired reads were assembled using Cufflinks 2 and genes.gtf also from hg19 annotation as a guide (Trapnell et al., 2012). Quantitation of transcripts was calculated as reported by Cufflinks in FPKMs and these were transformed to TPMs according to the methods described in Li et al, as previously described. Data structures using Bioconductor packages GRanges were used to unify the results.

### Example 2—Results

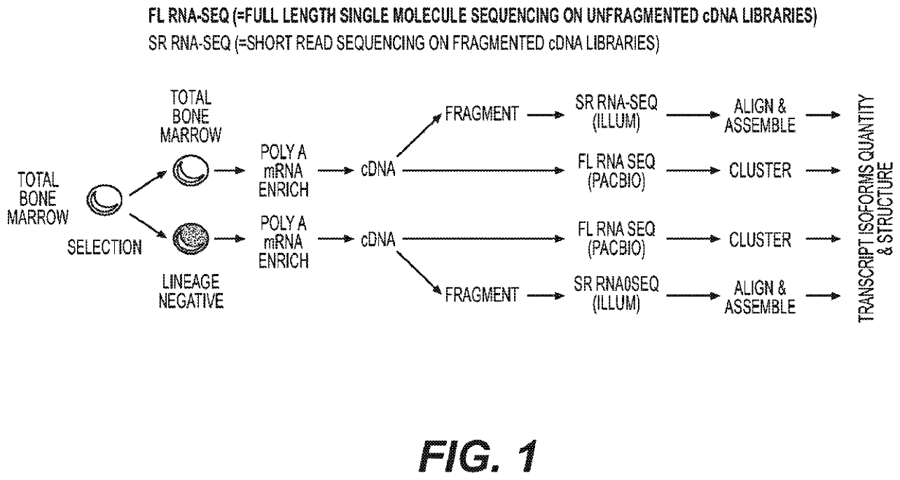

Total human bone marrow cell population (T) dominated by differentiated cells was compared with a small (<1%) subpopulation of lineage-negative progenitor cells (N) using single-molecule, full length RNA-seq (FIG. 1). Samples were also analyzed at 20 and 100 million read depths by conventional RNA-seq that relies on the computational assembly of transcripts from short read sequences of fragmented cDNAs. The representative focus was on the analysis of two representative genes that are abundantly expressed in hematopoietic cells (EEF1A1 & ANXA1) to provide details of the analysis. Utilizing complete transcriptomes generated either by full length or short read RNA-seq, the composite results are described and then a comparison with protein fragments identified by mass spectrometry is also provided.

Eukaryotic translation elongation factor 1 alpha 1 (EEF1A1) is a highly abundant, conserved protein that delivers aminoacyl-tRNAs to the ribosome during protein synthesis but has also been found to contribute to additional cellular functions. The EEF1A1 gene spans 5.2 kb on chromosome 6q13 and results in a 3.5 kb transcript that contains six protein coding exons (RefSeq HG19). Short read (short-read) RNA-seq of total and lineage-negative bone marrow populations matched to the known reference transcripts and contained the Canonical open reading frame (ST1-C, ST2-C*, red; SN1-C, blue). One of the transcript isoforms, however, did not include the long 3′ UTR (ST2-C*) and one novel isoform in lineage-negative cells (SN2-3) skipped four coding exons due to an alternative splice acceptor and did not contain the long 3′ UTR in the RefSeq data base.

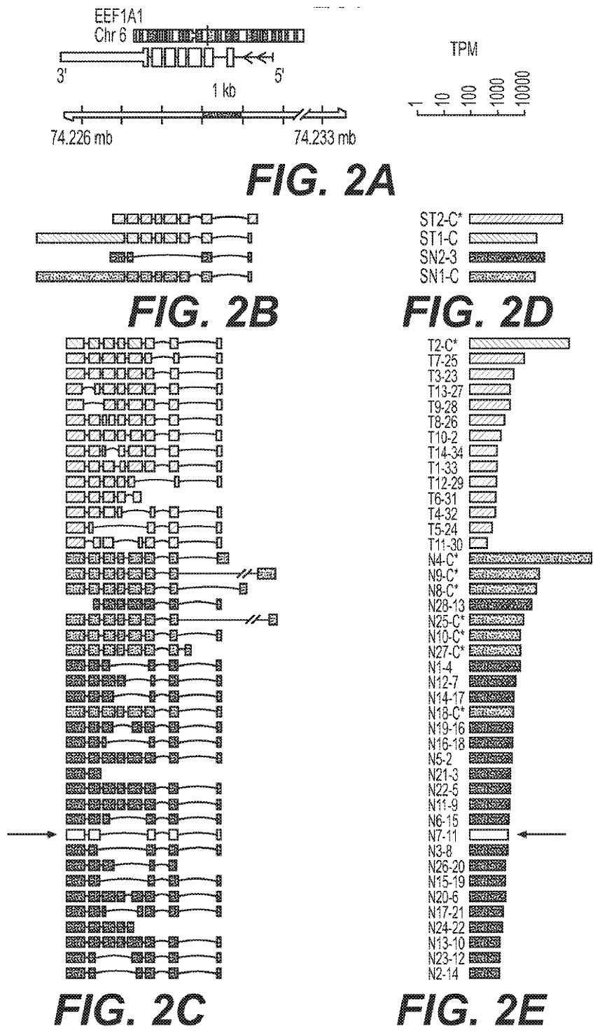

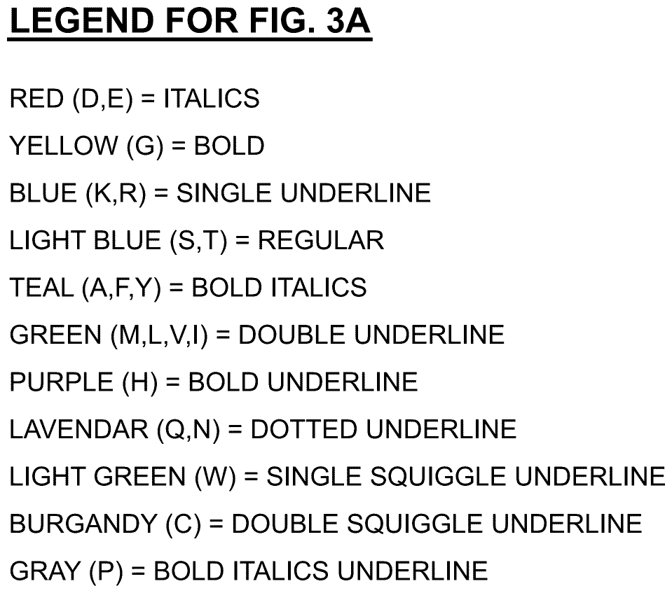

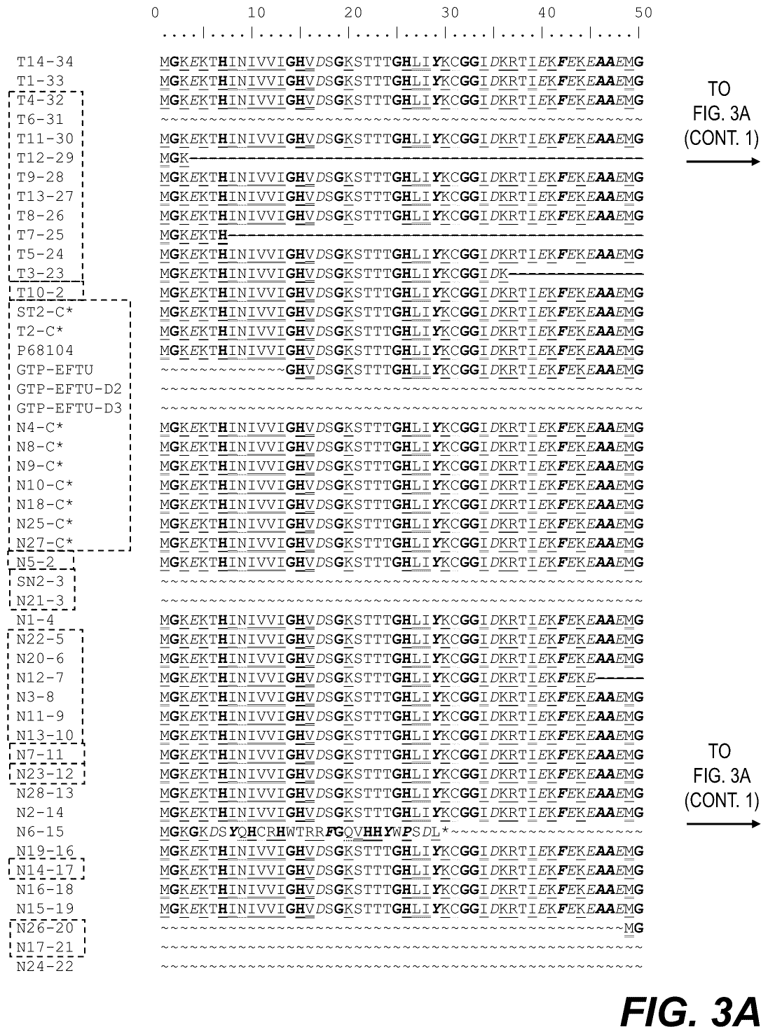

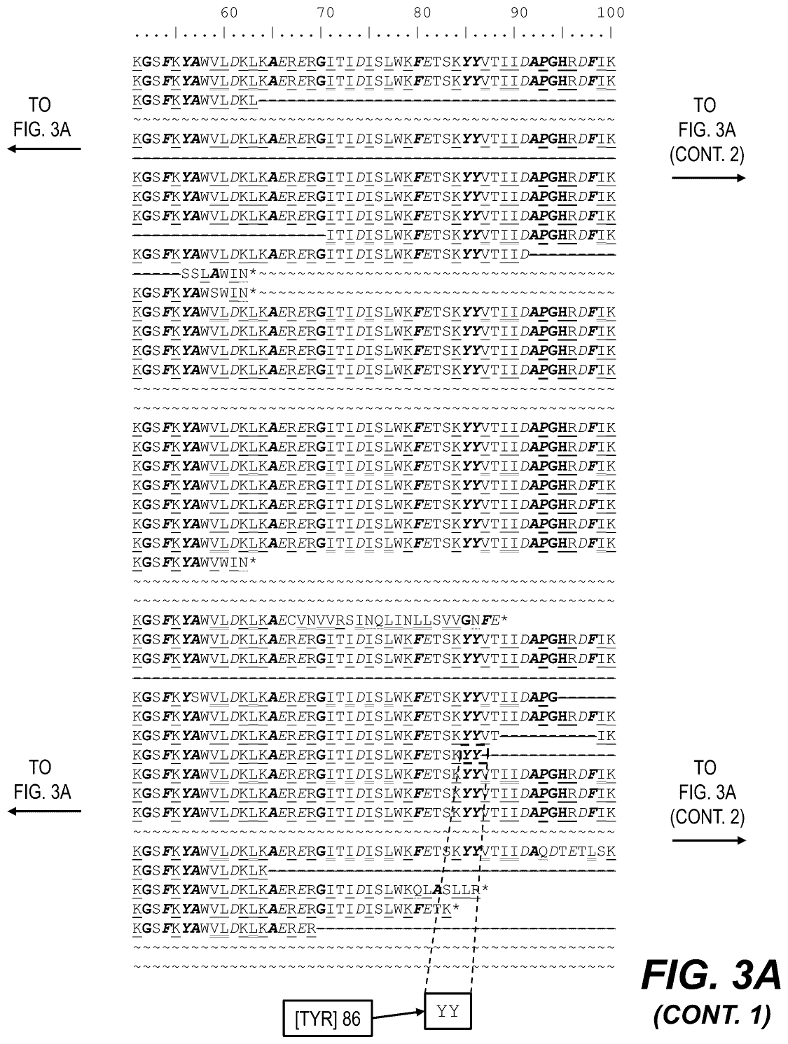

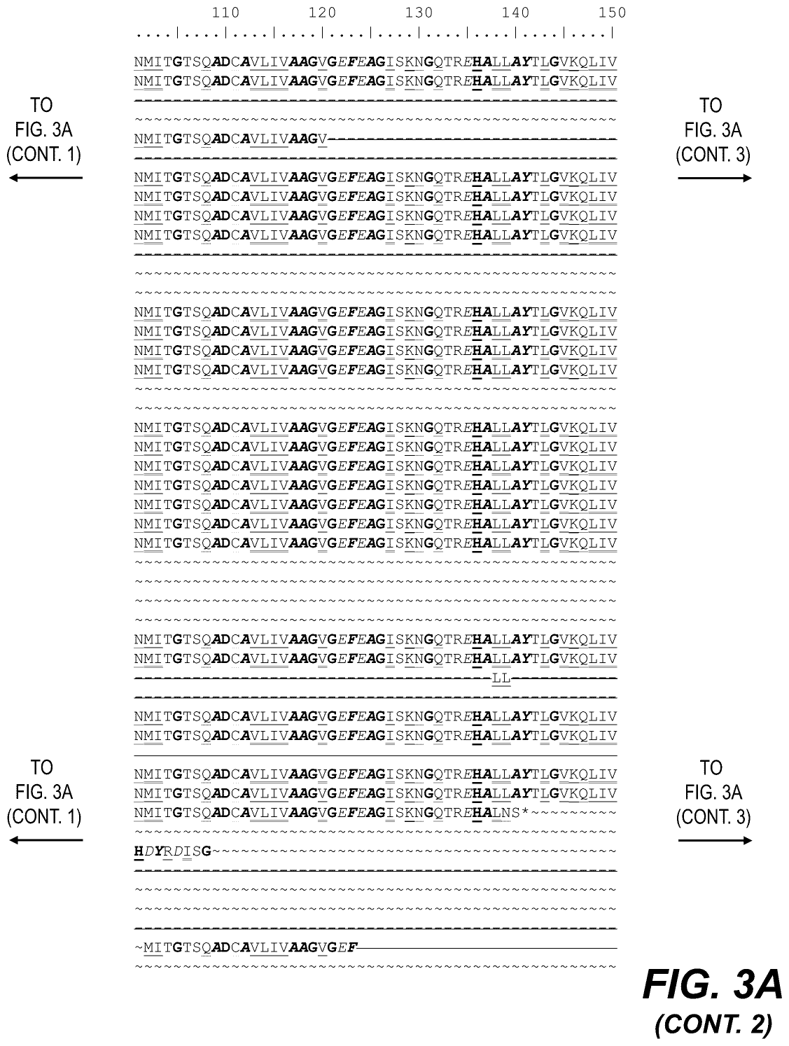

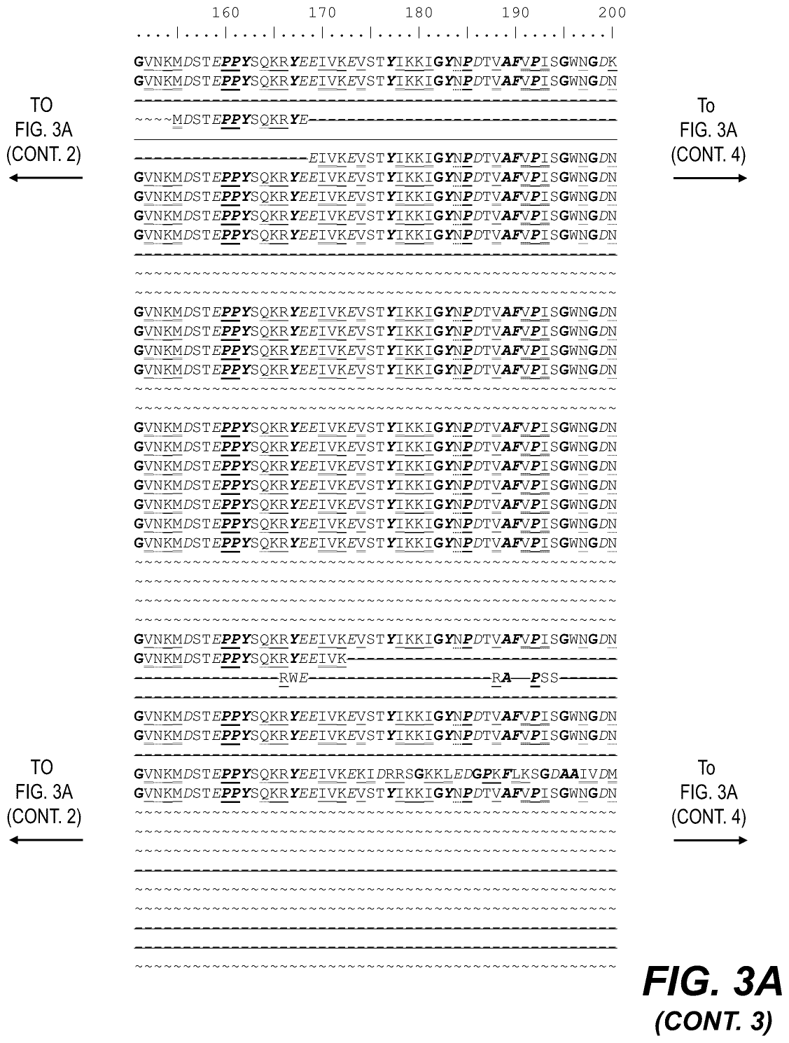

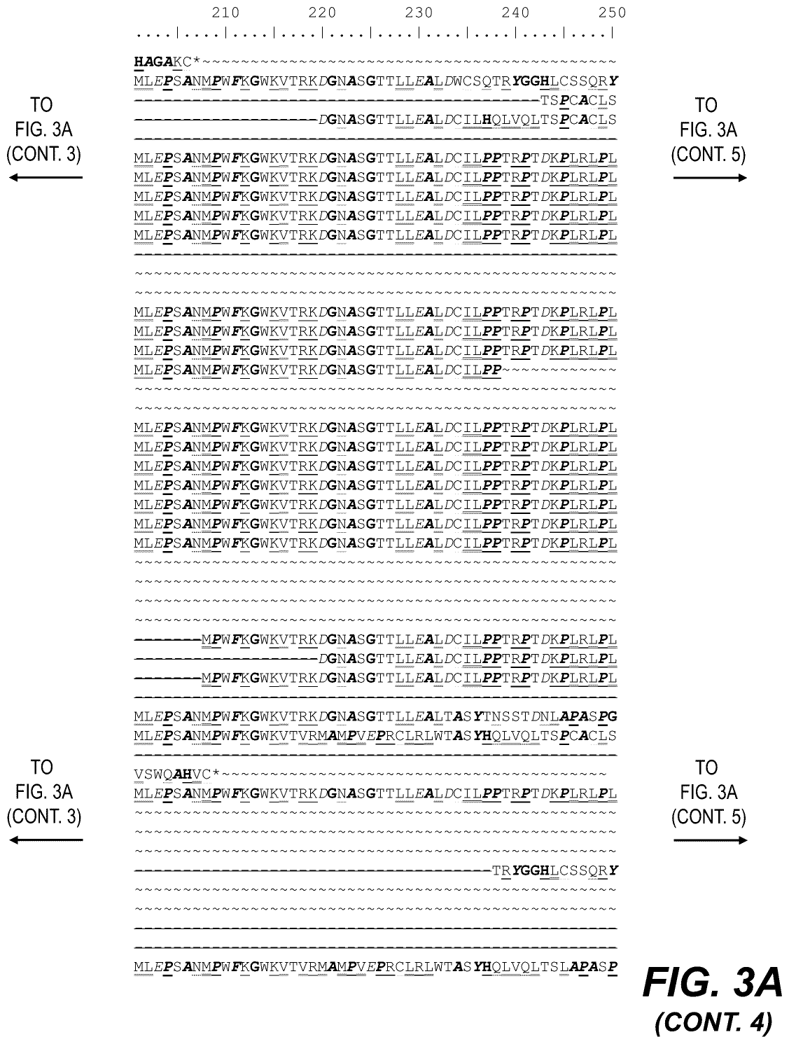

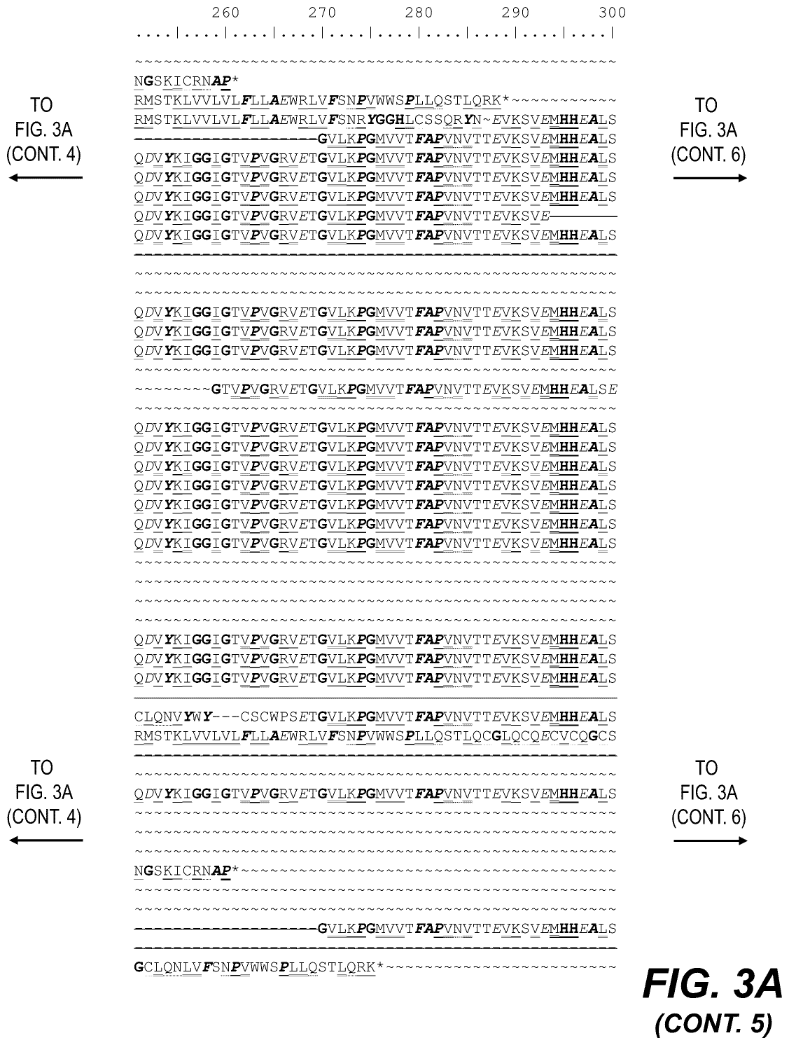

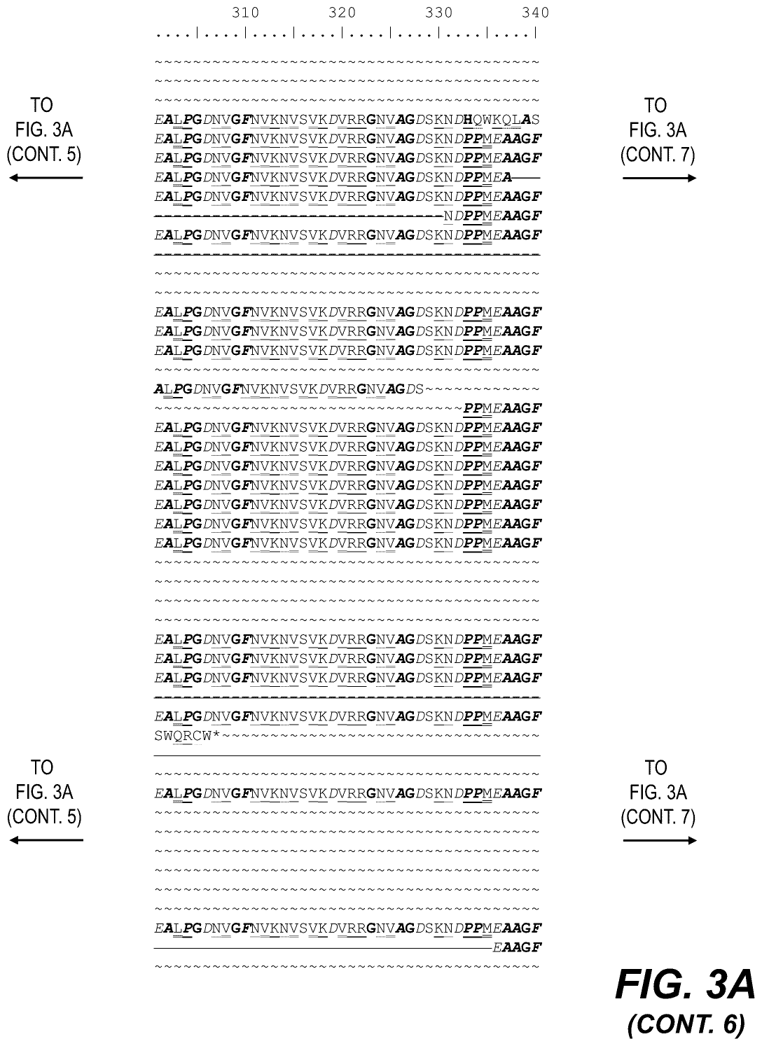

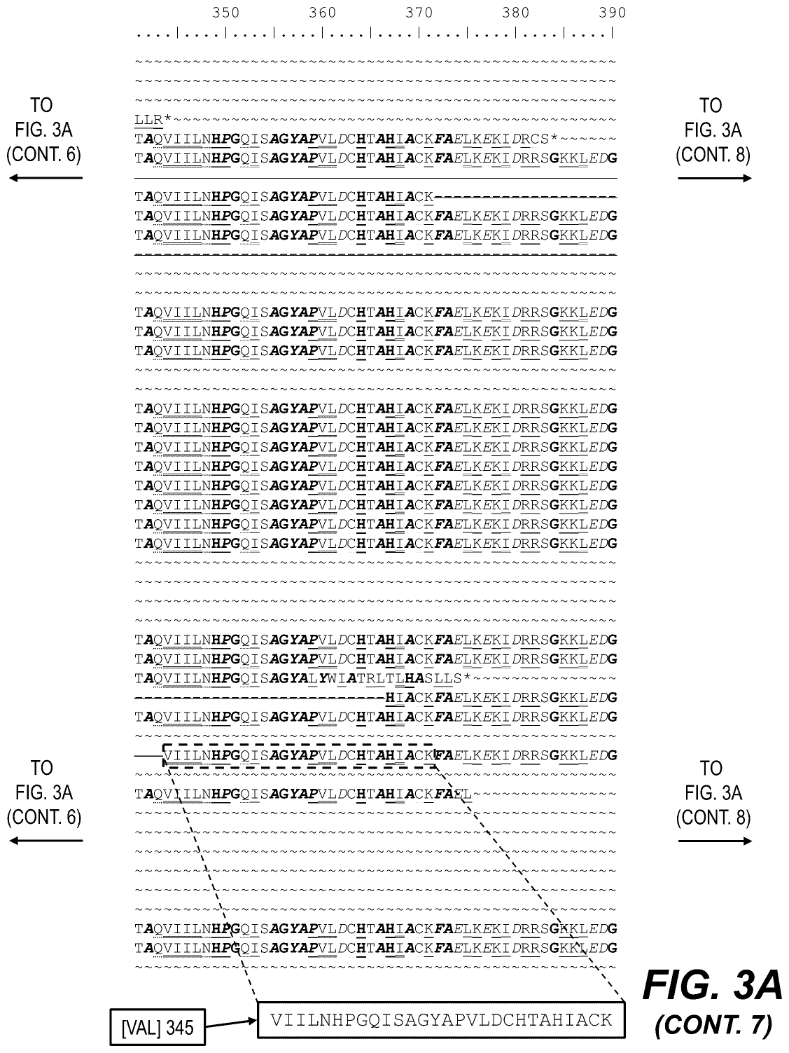

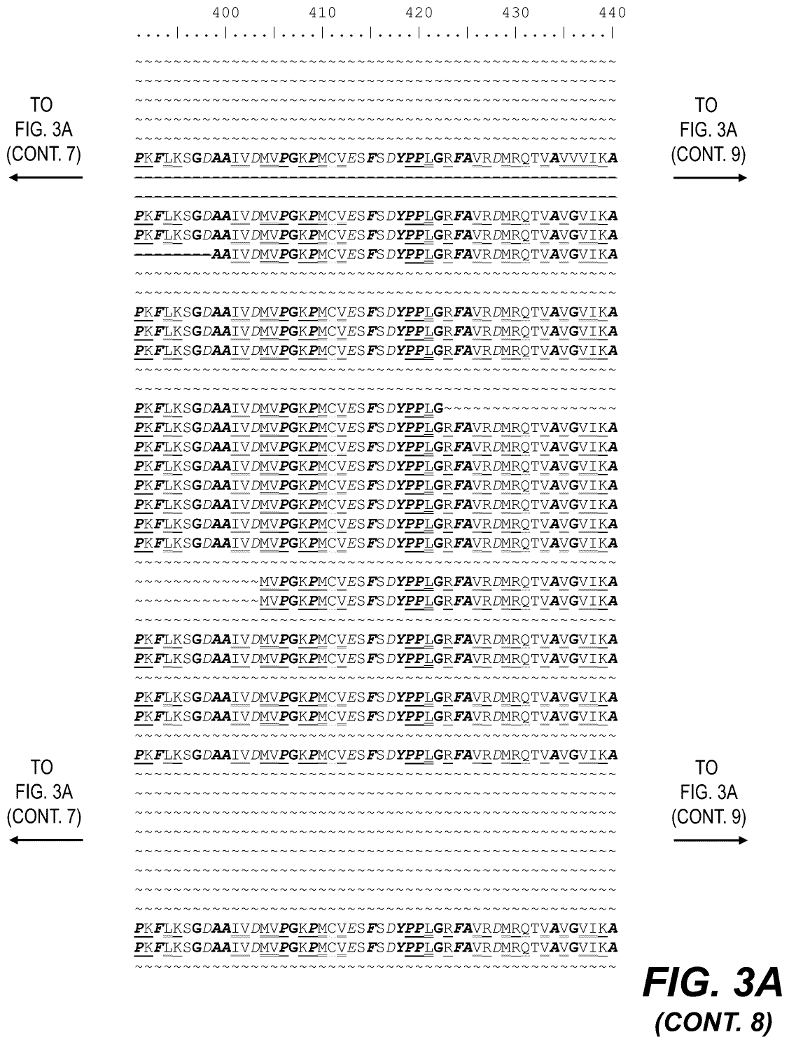

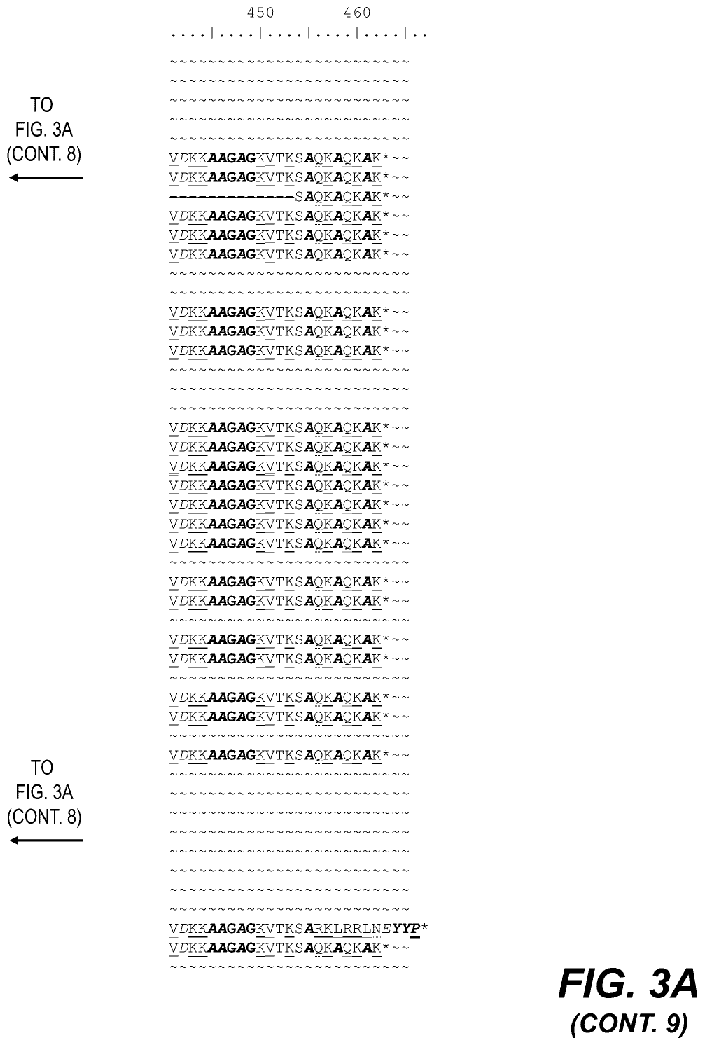

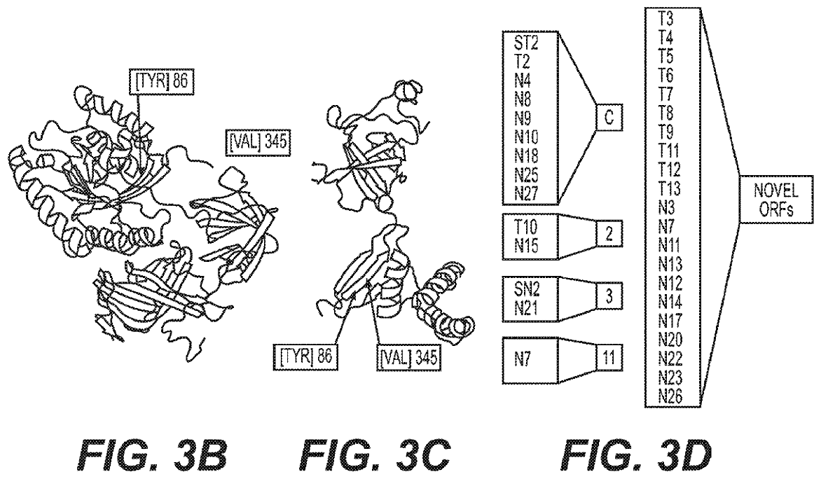

In contrast to this, the EEF1A1 transcript isoforms obtained from full length (long-read) RNA-seq (FIG. 2C) showed seven isoforms from lineage-negative (N4-C*, N9-C*, etc.) and one from the total bone marrow cell population (T2-C*) that contain the canonical open reading frame (P68104; see below FIG. 3A). An additional 34 novel transcript isoforms were identified, 14 of which were from the total and 28 from the lineage-negative cell populations. Exon-skipping was seen in 22, e.g., T9-28, exon-splitting in 2, T8-26, N13-10, and alternative donor/acceptor sites in 30 transcript isoforms, e.g., T7-25. No transcripts were found that contain the long 3′ UTR included in the Refseq data base (FIG. 2C). Transcript abundance showed a 16-fold range amongst the isoforms identified with short read RNA-seq whereas full length RNA-seq revealed an approximately 1,700-fold dynamic range across the different isoforms (FIG. 2D, FIG. 2E). From these observations, full length (long-read) RNA-seq can uncover novel transcript isoforms that distinguish bone marrow cell subpopulations and captures a much broader range of transcript abundance.

Open reading frames predicted from the transcript isoforms of EEF1A1 were aligned to the canonical protein and overlayed with its 3D structure (FIG. 3A, FIG. 3B). Out of the 21 novel protein isoforms predicted from the transcriptome analysis (FIG. 3A, FIG. 3D) the N7 protein contained a unique peptide that was distinct from fragments of the canonical EEF1A1 protein and detectable by mass spectrometry. The N7 transcript is typically found in the lineage-negative cell population only at low abundance (21 TPM; see FIG. 2E) and is predicted to code for a 205 aa protein that lacks the central 258 amino acids of the EEF1A1 protein and thus joins Y86 with V345 (FIG. 3A, FIG. 3C). A unique peptide spanning Y86 to V345 was detected by mass spectrometry analysis of tryptic digests of proteins extracted from lineage-negative cells (FIG. 3A) and thus confirms that the N7 transcript is translated into a protein expressed at sufficient levels to be detectable by mass spectrometry.

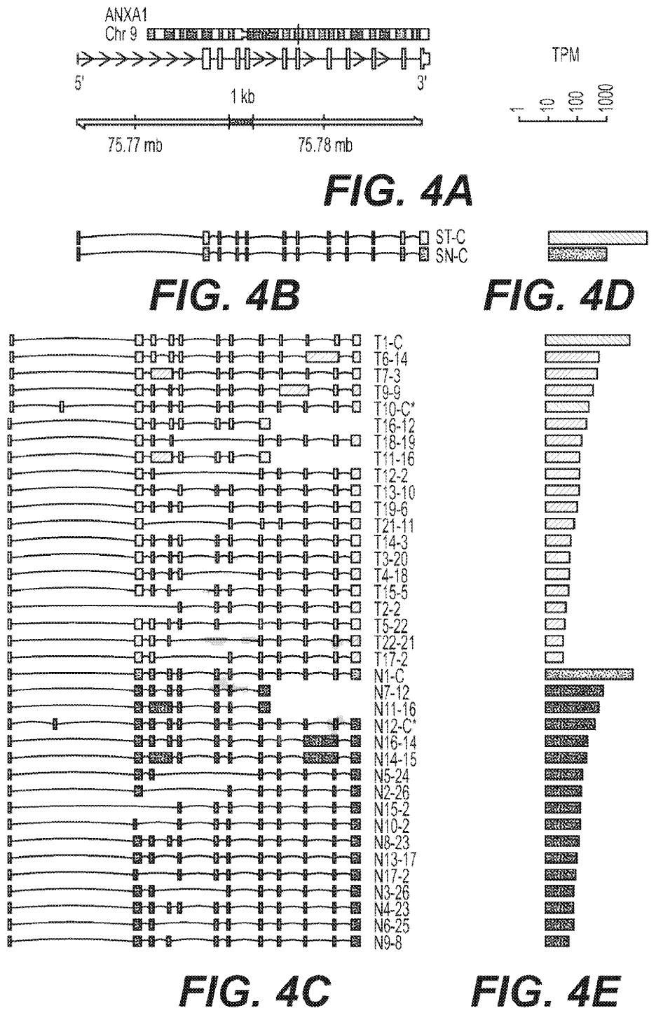

Annexins are known as organizers of membrane dynamics that include binding proteins for endocytosis, exocytosis, and other localization functions. The ANXA1 gene spans 18 kb on chromosome 9q21 with 12 coding exons and results in an approximately 1.5 kb transcript. With the conventional short read (short-read) RNA-seq and computational transcript reconstruction only the canonical ANXA1 transcript was found in total bone marrow and in lineage-negative cell populations. In contrast, full length (long-read) RNA-seq identified 36 transcript isoforms in addition to the canonical transcript (FIG. 4C): 21 in the total and 17 in the lineage-negative cell population. Two of these novel isoforms predict the canonical protein, P04083 (T10-C*, N12-C*), while others contain distinct open reading frames generated by exon-skipping (25 isoforms; e.g., T16-12, N7-12), alternative donor and acceptor sites (11 isoforms; e.g., T19-6, N10-2) and intron retentions (9 isoforms; e.g., T6-14, N11-13). The abundance of ANXA1 transcript expression was >10-fold lower in the lineage-negative cells when measured with short read RNA-seq (FIG. 4D). In contrast, full length RNA-seq revealed a 200-fold range of distinct expression levels of the different isoforms when comparing lineage-negative and total bone marrow cells (FIG. 4E).

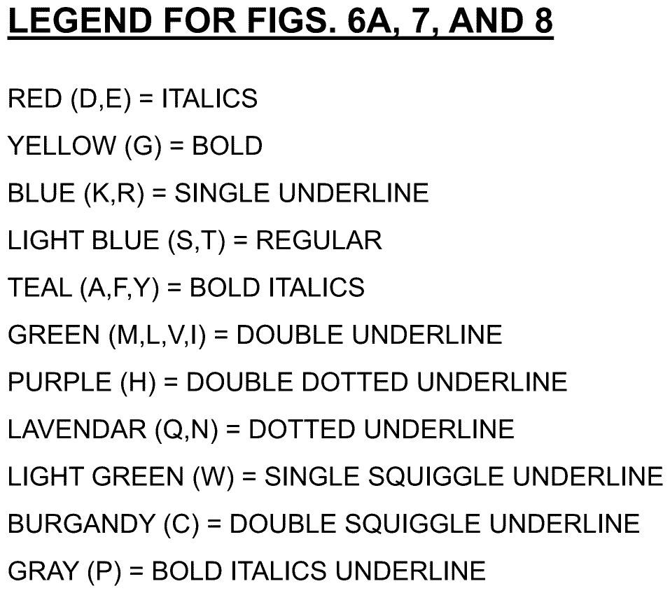

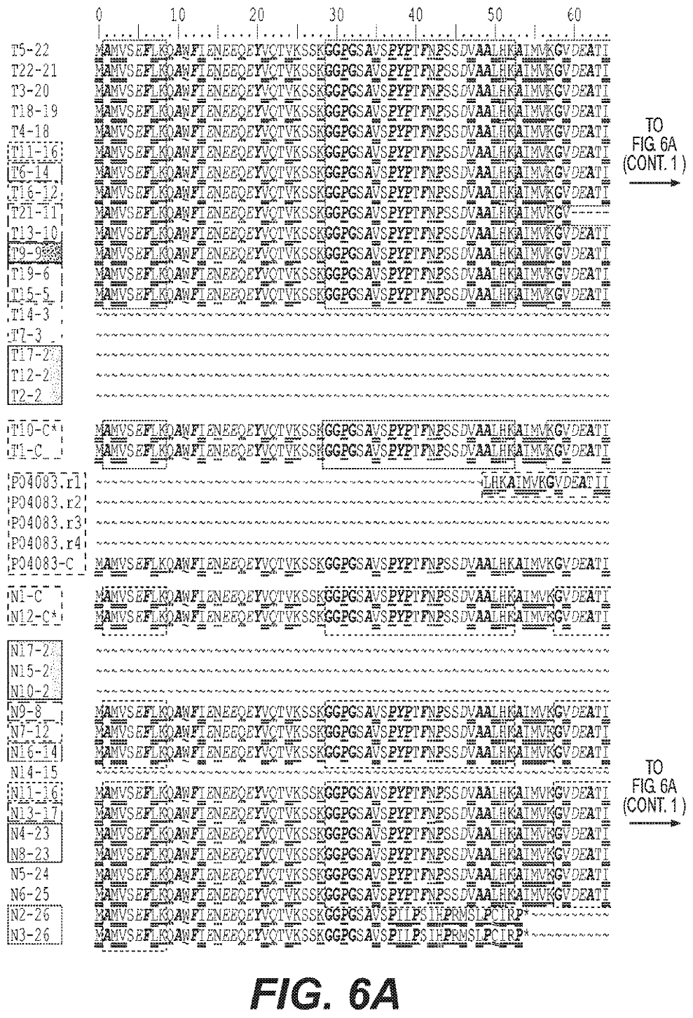

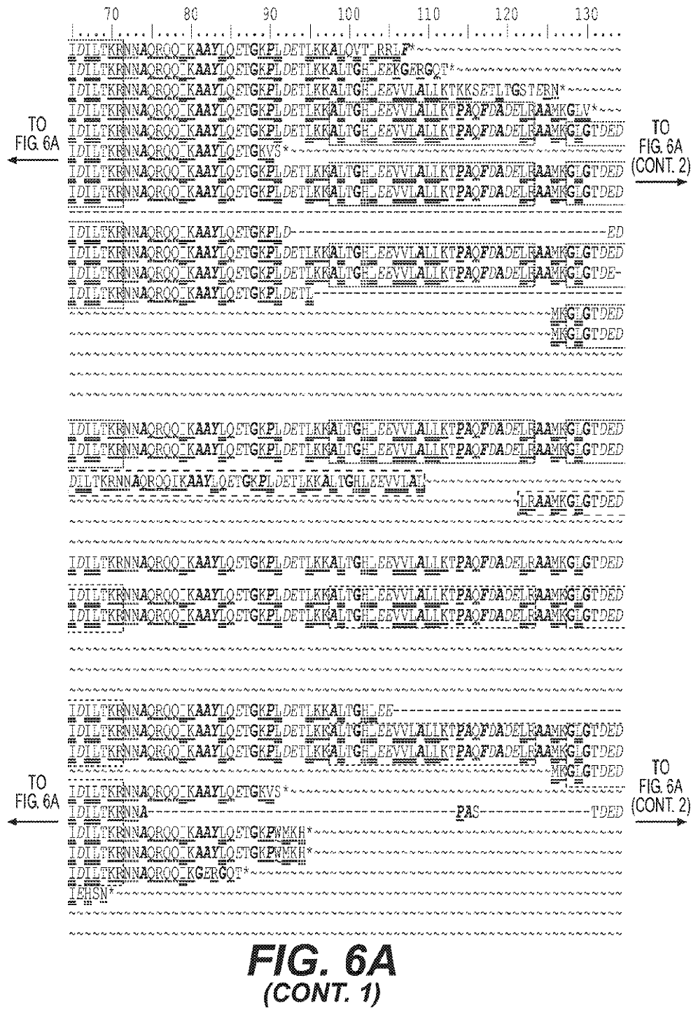

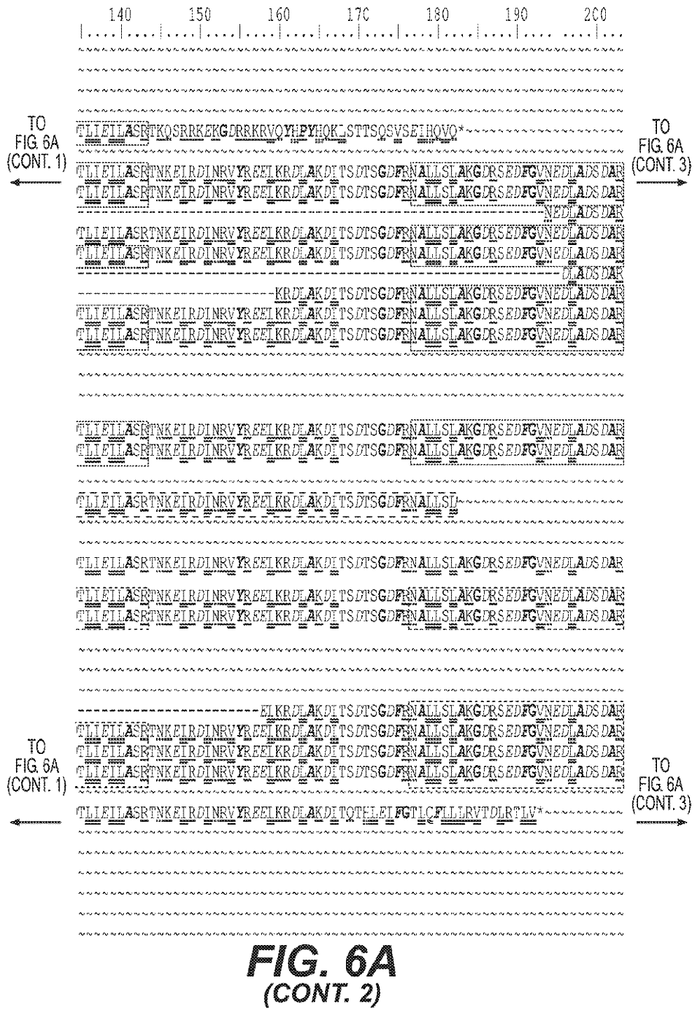

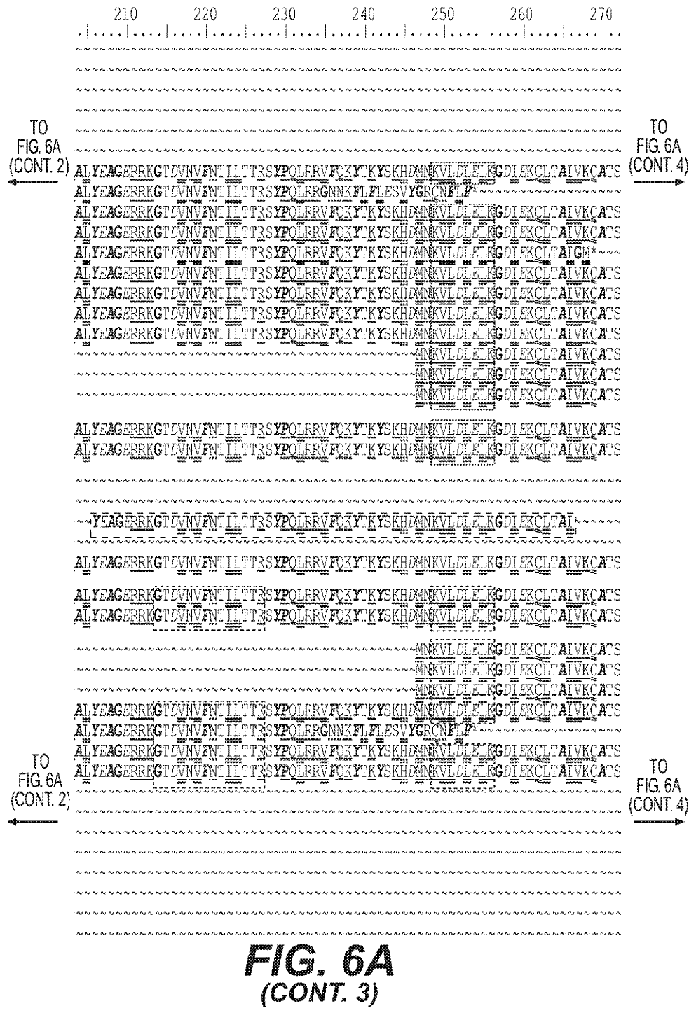

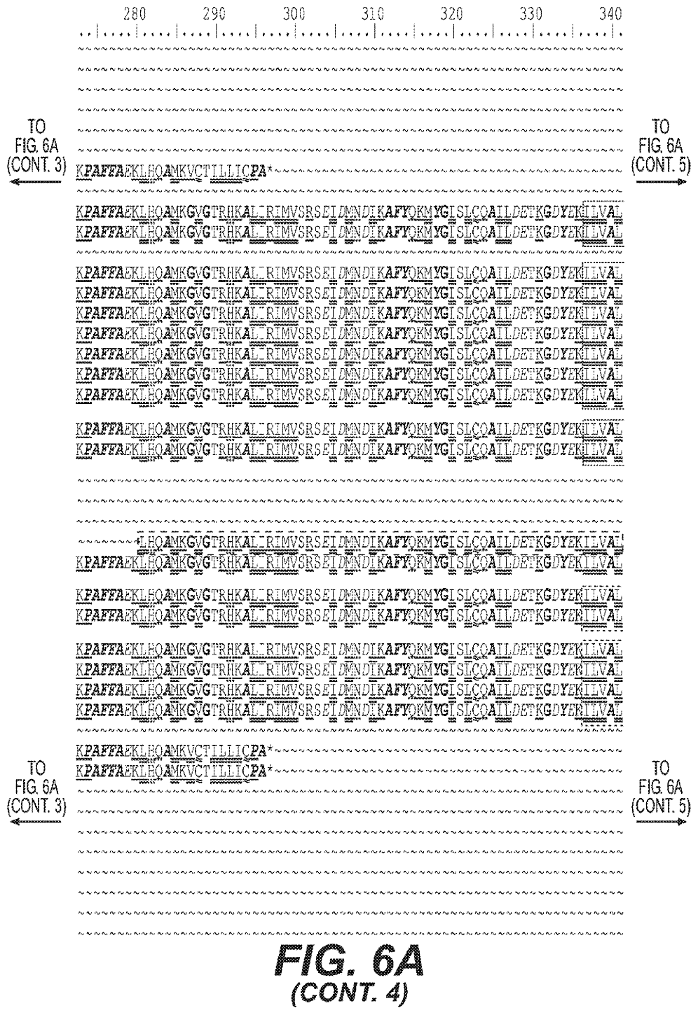

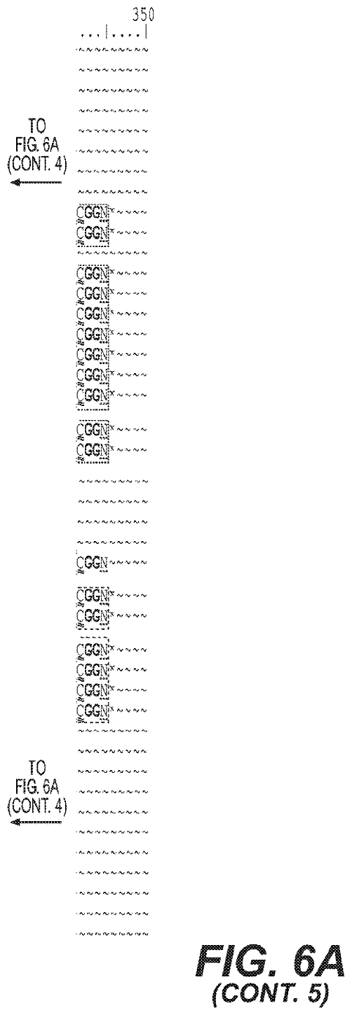

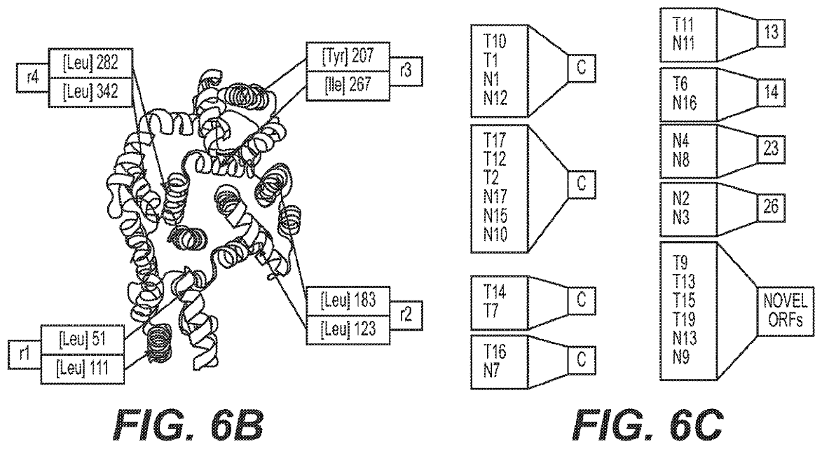

The canonical ANXA1 protein contains four repeat regions (r1-r4) of approximately 70 amino acids, each with a motif for calcium binding. The sequences are highlighted and the alignment of the predicted ORFs from transcript isoforms in total and lineage-negative bone marrow cells shows the overlaps with the canonical protein and with each other (FIG. 6A, FIG. 6B). Mass spec analysis of the proteins confirmed the presence of peptides in both cell populations (FIG. 6A).

The canonical ANXA1 protein is predicted by 4 different transcript isoforms (T1-C, T10-C*, N1-C and N12-C*). The T10-C* and N12-C* transcripts are structurally different from T1-C and N1-C, each containing a novel exon in the 5′ UTR (see FIG. 4c). ORF 2 matches with the r4 repeat and is contained in 7 different transcript isoforms whilst ORF 12, 13 and 14 matched with r1 to r3 repeats. These transcripts were detected in both cell populations. Additionally, ORF 3 (T14, T7), ORF 23 (N4, N8) and ORF 26 (N2, N3) are derived from two different transcript isoforms but typically found in only one of the cell populations (FIG. 6A, FIG. 6C). Finally, five novel exon-skipping proteins are predicted from transcript isoforms in total and lineage-negative cells (T13, T15, T19, N13 and N9). Full length (long rage) RNA-seq therefore shows a complex set of ANXA1 protein isoforms that distinguish bone marrow cell subpopulations.

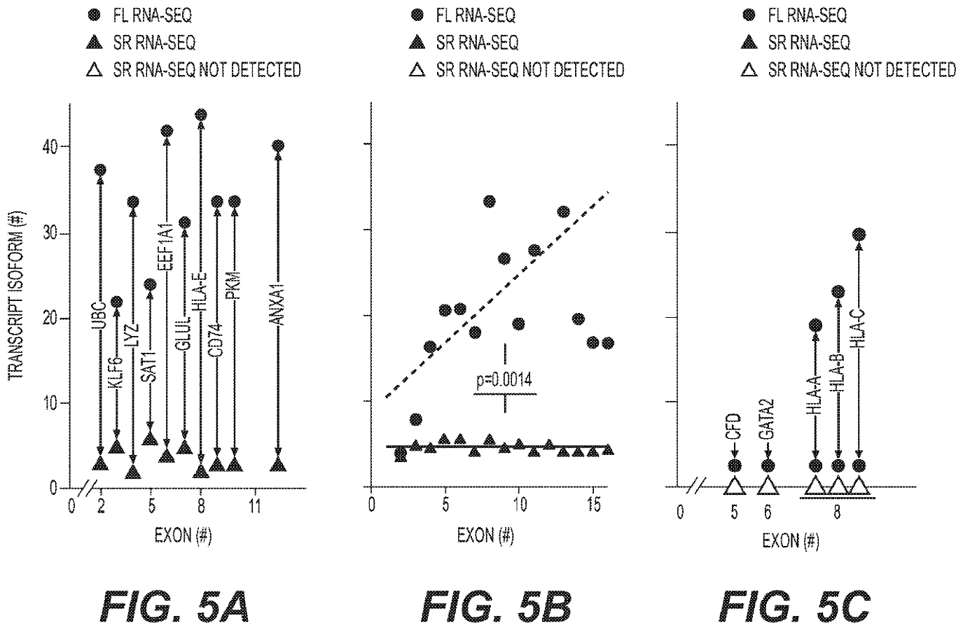

As described above for ANXA1 and EEF1A1, full length (long-read) RNA-seq of bone marrow cell populations revealed an approximately 10-fold higher number of transcript isoforms than found by conventional short read RNA-seq. To investigate whether this is true for other genes, genes with <6 coding exons (UBC, KLF6, LYZ, SAT1) were evaluated and it was found that conventional short-read RNA-seq only found 1 to 4 isoforms, whereas long-read RNA-seq identified 12 to 36 isoforms. This was similar at loci with >6 coding exons where only 1 to 3 versus 31 to 43 transcript isoforms were identified by the two different RNA-seq approaches (FIG. 5A).

The analysis was extended by arranging genes by mean exon number and identifying the loci with the top five transcript isoform counts in each bin. The number of transcript isoforms identified by short read RNA-seq surprisingly plateaued at four isoforms irrespective of the number of exons in a given gene (FIG. 5B). In contrast, full length RNA-seq showed an increase in transcript isoform number with increasing complexity of genomic loci as indicated by their number of canonical exons (p=0.0014). An increase in sequencing depth for short read RNA-seq from 20 to 100 million reads did not impact this maximum significantly (p>0.05). Thus, the analysis supports the notion that the complexity of the transcriptome is underestimated by short-range RNA-seq irrespective of the complexity of genomic loci evaluated.

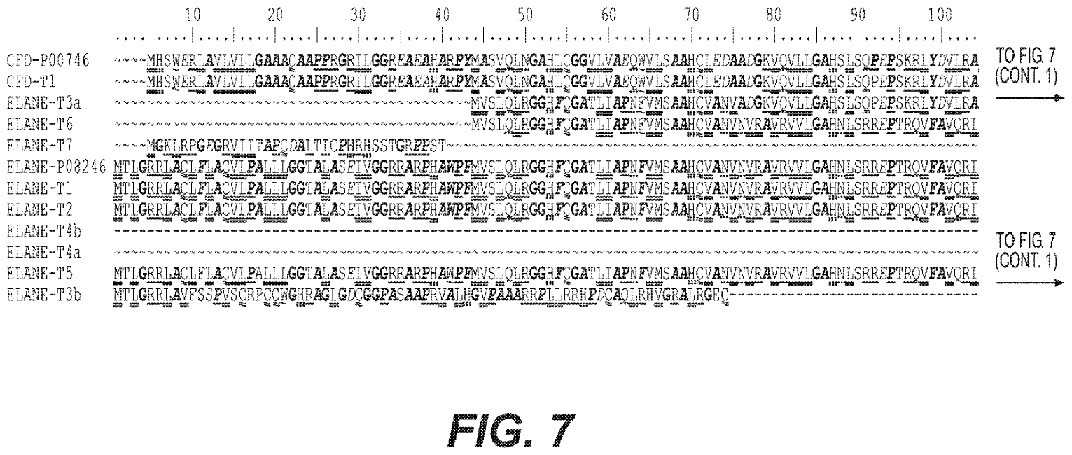

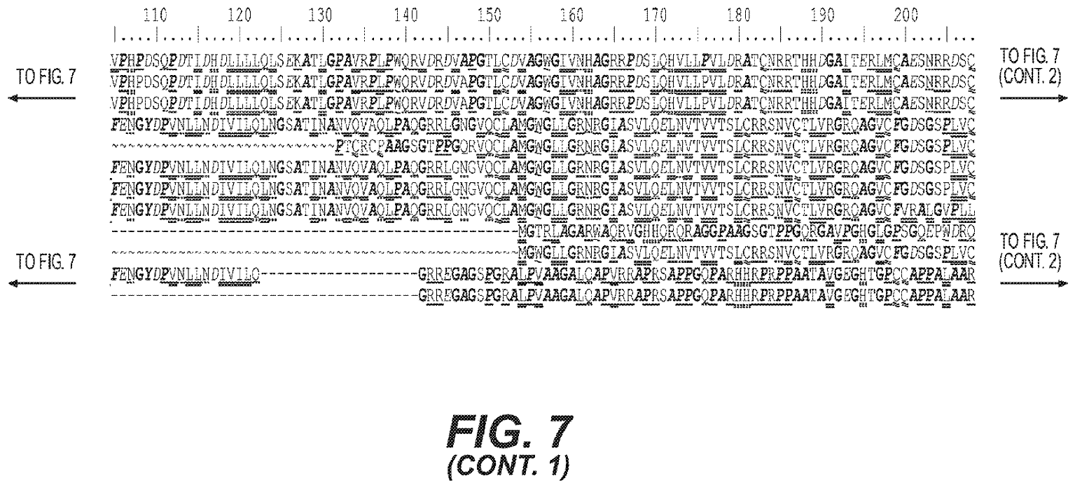

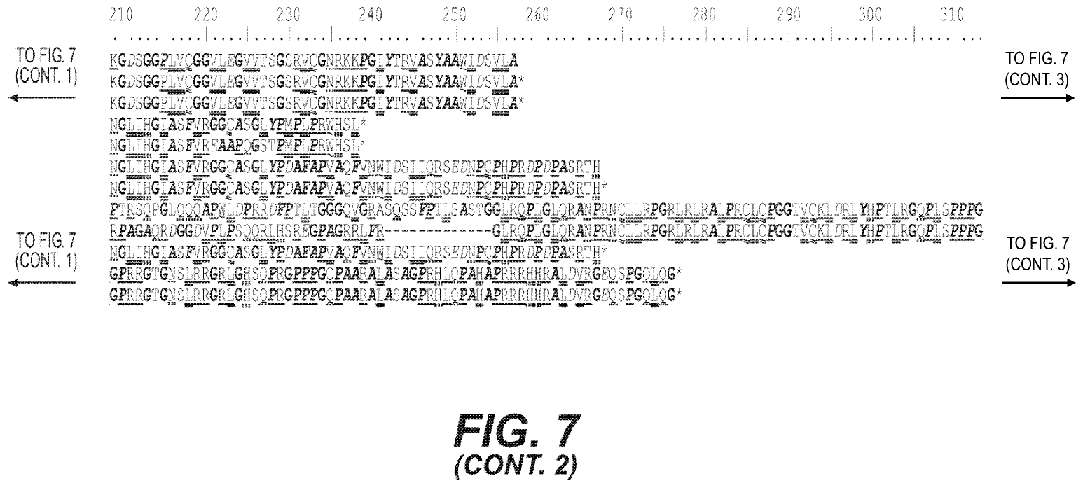

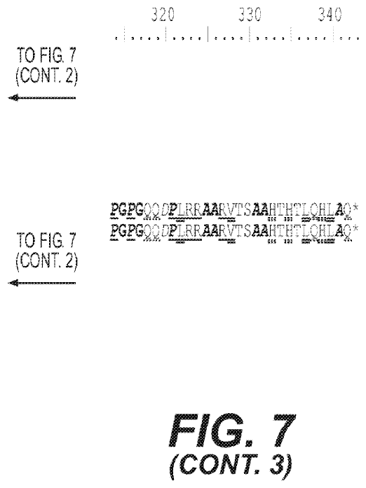

Additionally, full length (long-read) RNA-seq identified transcripts for CFD, GATA2, HLA-A, -B and -C that were missed by short read RNA-seq (FIG. 5C). The inability to detect or assign transcripts for these loci with short read RNA-seq may be explained by the paralogous nature of the genes involved: CFD is located on chr 19 with AZU1, PRTN3 and ELANE. These four genes rank second in the list of regions of homozygosity coldspots on human autosomes. Genes located in this run of homozygosity (ROH) region are under evolutionary pressure to remain heterozygous. Additionally, the ELANE and CFD proteins are 78% homologous (FIG. 7; canonical proteins).

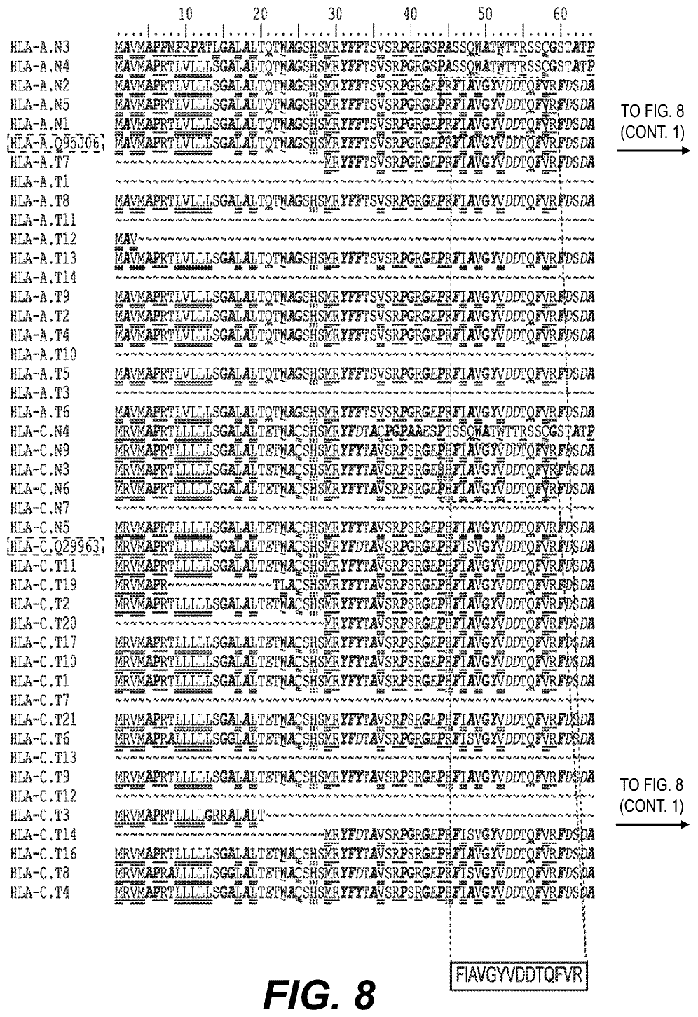

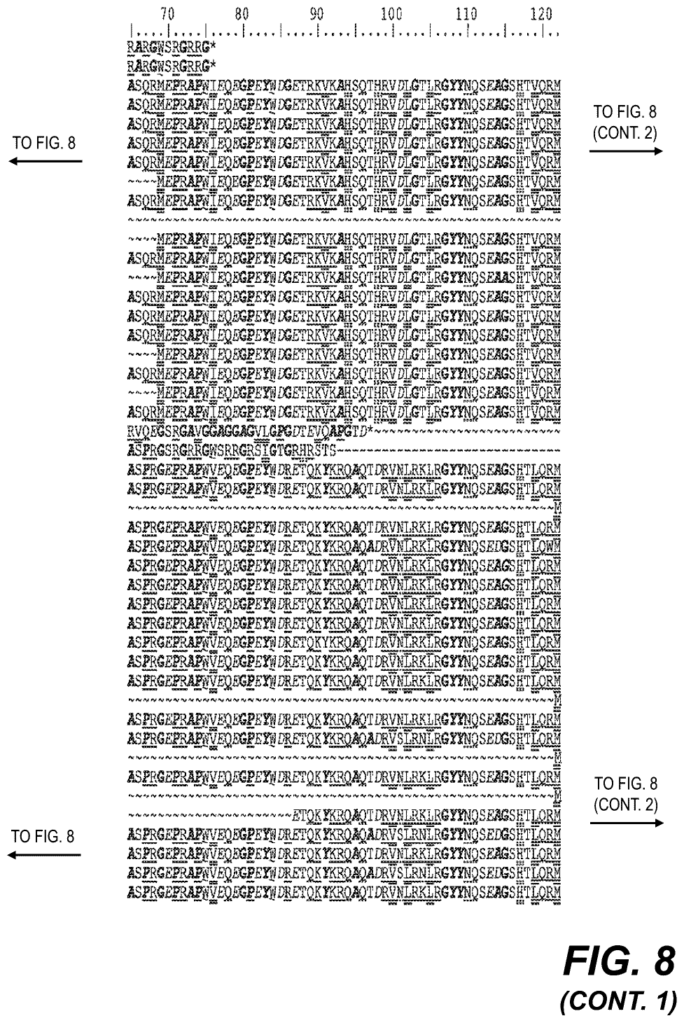

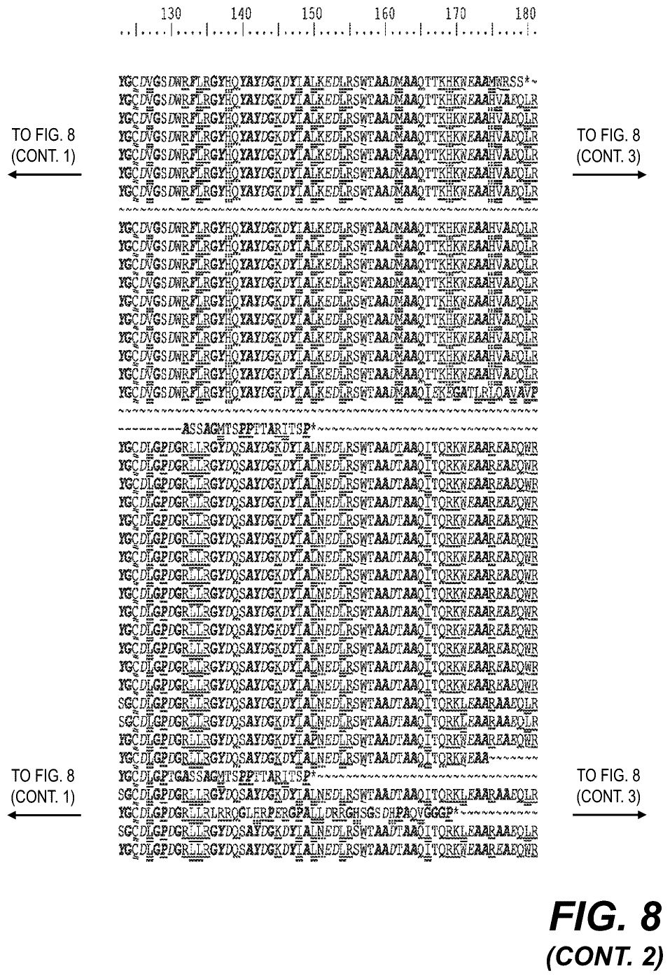

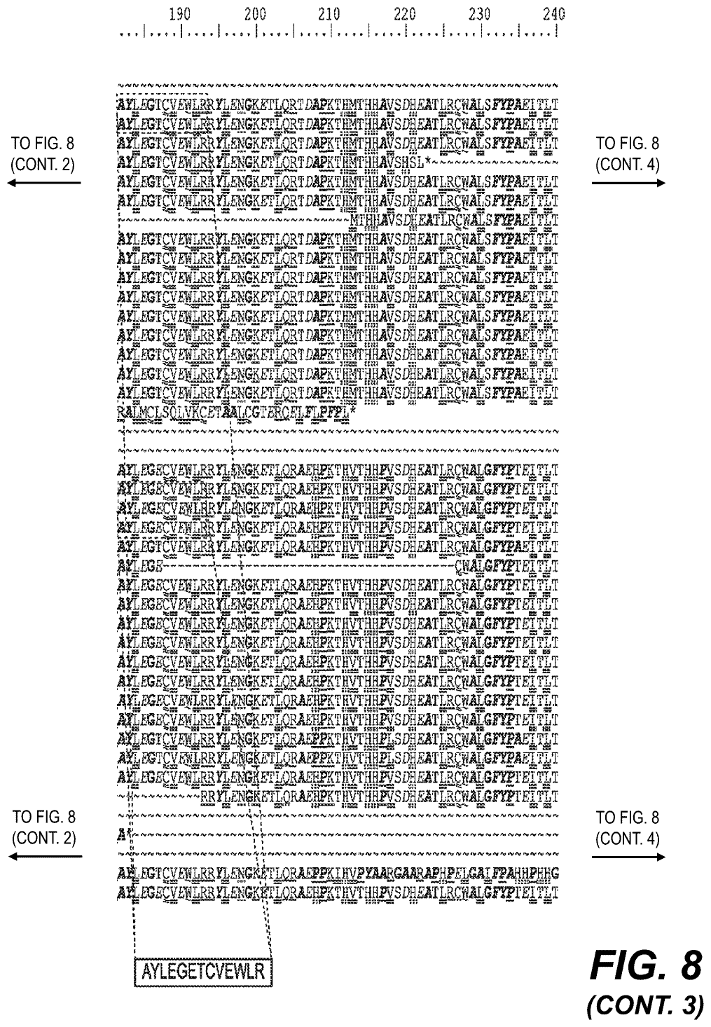

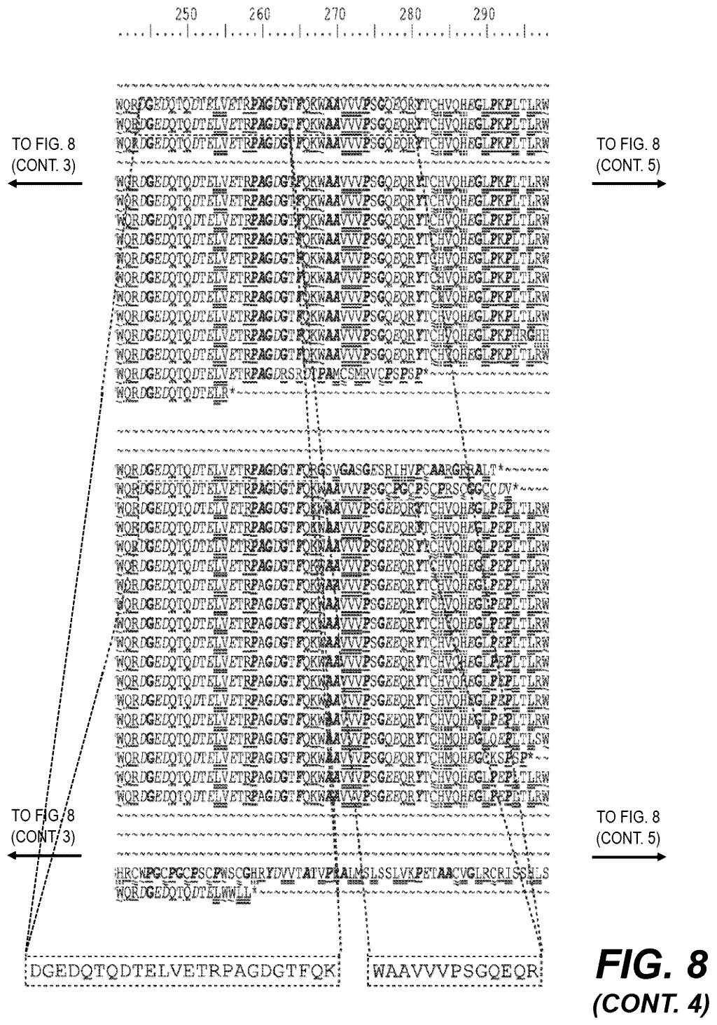

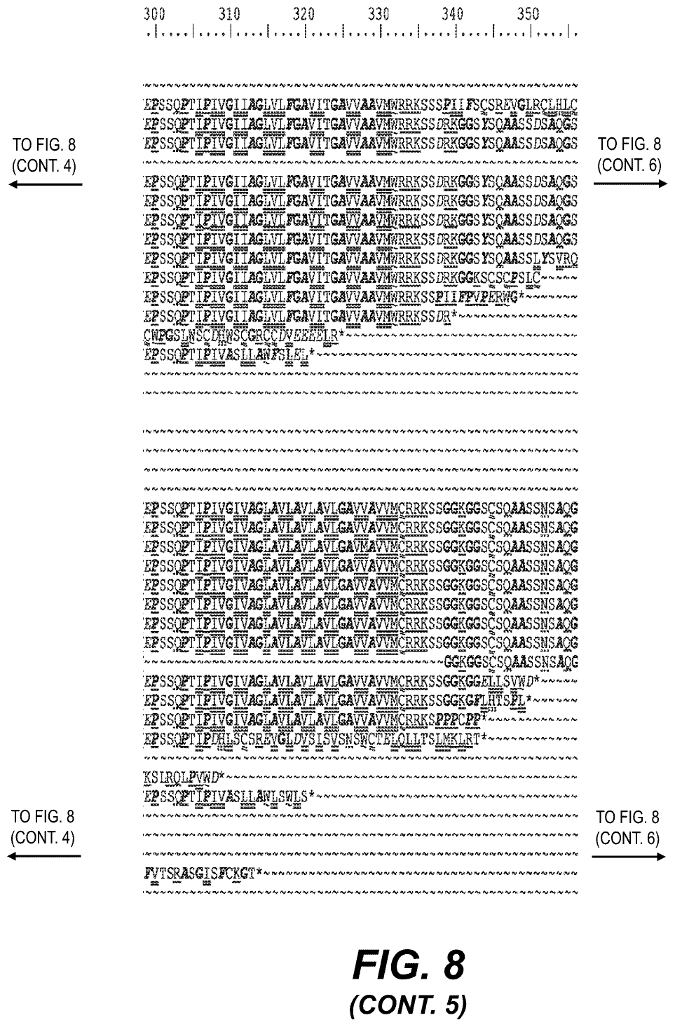

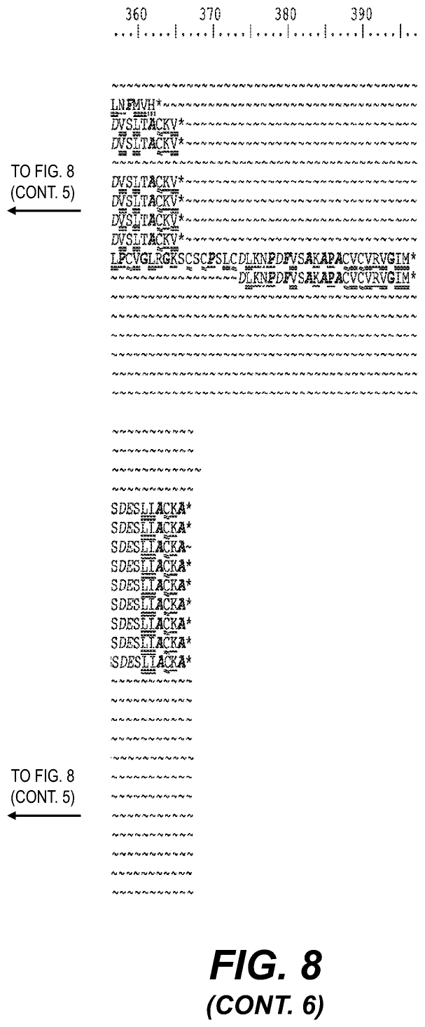

Short read RNA-seq uncovered transcripts from three of these four genes, but missed CFD, which is not surprising, because blastn analysis matched CFD fragment sequences to ELANE. Using full length RNA-seq, unique isoforms were identified for each of the genes in these regions of homozygosity. Similarly, HLA-A, HLA-B, and HLA-C are paralogs with >80% identity that cannot be mapped appropriately and detected by short read RNA-seq (FIG. 5C and FIG. 8). These data suggest that the presence of transcripts from paralogs adds to the complexity of alignments and obfuscates transcript reconstruction from short reads as well as the estimate of transcript abundance.

To assess the biological relevance of the transcript isoforms found and their predicted open reading frames, detectable proteins were identified in the bone marrow cell populations by mass spectrometry. A range of genomic loci that contain between 2 and 16 exons was assessed and associated with the highest number of transcript isoforms. Peptides were confirmed for 52 of the 150 transcripts depicted in FIG. 5B.

As mentioned above, HLA-A, -B and -C transcript isoforms were only detected by full length RNA-seq (FIG. 5C). It is noteworthy that mass spectrometry identified 4 distinct peptides predicted from the HLA-A and HLA-C transcript isoforms that were detected (FIG. 8). This supports the relevance of these transcripts and the ORFs derived from the long-read RNA-sequencing.

For long-read RNA-sequencing, 56% of the transcripts mapped to loci with 4 or more exons and 31% mapped to loci with 8 or more exons. In contrast, short-range RNA sequencing mapped only 13% of transcripts to loci with >4 and 5% with >8 exons. Thus, full length RNA-seq provides a significant (p<0.0001) 4 to 6-fold gain in information. Given that over half of the detected transcripts are from multi-exon genes, the ability to span 2 exons with short reads may be inadequate to resolve a full length transcript successfully without the addition of longer reads. Also, ambiguous mapping of the short reads explains the high number of transcripts being mapped to genes with 1 or 2 exons. The data here also shows that short-read RNA sequencing reaches a maximum of approximately four transcript isoform even for complex loci 8, whereas long-read RNA sequencing shows a significant increase in transcript isoforms with increasing complexity of genomic loci (FIG. 5B).

To validate the sequences of the transcript isoforms described above, unfiltered raw reads from the short-read RNA sequencing were used. This complete set of raw reads was mapped against each of the respective transcript isoforms described above. Short reads with a full 100 base pair coverage were blasted against the individual transcript isoforms obtained from long-read RNA sequencing. Beyond that, long-read RNA sequencing with independent samples was used for additional validation of isoform reads not covered by the short-read RNA sequencing. From these approaches, 83% of the transcript isoforms reported above were confirmed (range 74% to 100%).

1-12. (canceled)

13. A method of monitoring progression of an abnormal condition in a subject in need thereof, the method comprising determining in the subject at two or more time points a level of a neopeptide that is a marker of abnormal cells associated with the abnormal condition, wherein the neopeptide is identified by:
a) sequencing long-read messenger RNA (mRNA) isolated from a first subset of a population of the abnormal cells,
b) identifying splice variants generated from the sequenced long-read mRNA,
c) determining in a second subset of the population of cells presence of neopeptides that are translated from the identified splice variants and determining the presence of the neopeptide in a population of normal cells,
wherein a single identified splice variant translates to a single neopeptide, and
wherein the presence of the neopeptide in the second subset of the population of abnormal cells and the absence of the neopeptide in the normal cells is indicative that the neopeptide is a marker for the abnormal cells.

14. The method of claim 13, further comprising determining whether the level of the neopeptide in the subject increased between the two or more time points, wherein an increase in the levels of the neopeptide is indicative of progression of the abnormal condition.

15. The method of claim 13, wherein the long-read mRNA is at least 70 nucleotides in length.

16. The method of claim 13, wherein the isolated mRNA is cytosolic mRNA.

17. The method of claim 13, wherein the population of abnormal cells is a population of cancer cells.

18. The method of claim 17, wherein the population of normal cells is a population of non-cancer cells of the same tissue origin as the population of cells.

19. The method of claim 13, wherein determining in the subject the level of the neopeptide comprises measuring presence of a binding agent that binds to the neopeptide.

20. A method of monitoring the efficacy of treatment of an abnormal condition in a subject in need thereof, the method comprising determining in the subject at two or more time points a level of a neopeptide that is a marker of abnormal cells associated with the abnormal condition, wherein the neopeptide is identified by:
a) sequencing long-read messenger RNA (mRNA) isolated from a first subset of a population of the abnormal cells,
b) identifying splice variants generated from the sequenced long-read mRNA,
c) determining in a second subset of the population of cells presence of neopeptides that are translated from the identified splice variants and determining the presence of the neopeptide in a population of normal cells,
wherein a single identified splice variant translates to a single neopeptide, and
wherein the presence of the neopeptide in the second subset of the population of abnormal cells and the absence of the neopeptide in the normal cells is indicative that the neopeptide is a marker for the abnormal cells.

21. The method of claim 20, further comprising determining whether the level of the neopeptide in the subject decreased between the two or more time points, wherein a decrease in the levels of the neopeptide is indicative that the treatment is effective.

22. The method of claim 20, wherein the long-read mRNA is at least 70 nucleotides in length.

23. The method of claim 20, wherein the isolated mRNA is cytosolic mRNA.

24. The method of claim 20, wherein the population of abnormal cells is a population of cancer cells.

25. The method of claim 24, wherein the population of normal cells is a population of non-cancer cells of the same tissue origin as the population of cells.

26. The method of claim 20, wherein determining in the subject the level of the neopeptide comprises measuring presence of a binding agent that binds to the neopeptide.

27. A method of identifying abnormal tissue in a subject, the method comprising detecting presence in a tissue sample from the subject of a neopeptide that is a marker of abnormal cells, wherein the neopeptide is identified by:
a) sequencing long-read messenger RNA (mRNA) isolated from a first subset of a population of the abnormal cells,
b) identifying splice variants generated from the sequenced long-read mRNA,
c) determining in a second subset of the population of cells presence of neopeptides that are translated from the identified splice variants and determining the presence of the neopeptide in a population of normal cells,
wherein a single identified splice variant translates to a single neopeptide, and
wherein the presence of the neopeptide in the second subset of the population of abnormal cells and the absence of the neopeptide in the normal cells is indicative that the neopeptide is a marker for the abnormal cells,
wherein the presence of the neopeptide in the tissue sample is indicative that the tissue is abnormal.

28. The method of claim 27, wherein the long-read mRNA is at least 70 nucleotides in length.

29. The method of claim 27, wherein the isolated mRNA is cytosolic mRNA.

30. The method of claim 27, wherein the population of abnormal cells is a population of cancer cells.

31. The method of claim 30, wherein the population of normal cells is a population of non-cancer cells of the same tissue origin as the population of cells.

32. The method of claim 27, wherein determining in the subject the presence of the neopeptide comprises measuring presence of a binding agent that binds to the neopeptide.


SEQUENCE LISTING (122 sequences)

SEQ ID NO: 1 - protein, 206 aa, Homo sapiens [sequence omitted]

SEQ ID NO: 2 - protein, 260 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDWCSQTRYGGHLCSSQRYNGSKICRNAP

SEQ ID NO: 3 - protein, 109 aa, Homo sapiens
MGKEKTHINIVVIGHVDSGKSTTTGHLIYKCGGIDKRTIEKFEKEAAEMGKGSFKYAWVLDKLTSPCACLSRMSTKLVVLVLFLLAEWRLVFSNPVWWSPLLQSTLQRK

SEQ ID NO: 4 - protein, 137 aa, Homo sapiens
MDSTEPPYSQKRYEDGNASGTTLLEALDCILHQLVQLTSPCACLSRMSTKLVVLVLFLLAEWRLVFSNRYGGHLCSSQRYNEVKSVEMHHEALSEALPGDNVGFNVKNVSVKDVRRGNVAGDSKNDHQWKQLASLLR

SEQ ID NO: 5 - protein, 284 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVMLEPSANMPWFKGWKVTRKDGNASGTTLLEALDCILPPTRPTDKPLRLPLGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRCS

SEQ ID NO: 6 - protein, 297 aa, Homo sapiens [sequence omitted]

SEQ ID NO: 7 - protein, 359 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAVDKKAAGAGKVTKSAQKAQKAK

SEQ ID NO: 8 - protein, 380 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKSAQKAQKAK

SEQ ID NO: 9 - protein, 425 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVENDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 10 - protein, 399 aa, Homo sapiens [sequence omitted]

SEQ ID NO: 11 - protein, 155 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAAIVDMVPGKPMCVESFSDYPPLGRFAVRDMRQTVAVGVIKAVDKKAAGAGKVTKSAQKAQKAK

SEQ ID NO: 12 - protein, 43 aa, Homo sapiens [sequence omitted]

SEQ ID NO: 13 - protein, 62 aa, Homo sapiens [sequence omitted]

SEQ ID NO: 14 - protein, 462 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 15 - protein, 225 aa, Homo sapiens
GHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPP

SEQ ID NO: 16 - protein, 70 aa, Homo sapiens
GTVPVGRVETGVLKPGMVVTFAPVNVTTEVKSVEMHHEALSEALPGDNVGFNVKNVSVKDVRRGNVAGDS

SEQ ID NO: 17 - protein, 90 aa, Homo sapiens
PPMEAAGFTAQVIILNHPGQISAGYAPVLDCHTAHIACKFAELKEKIDRRSGKKLEDGPKFLKSGDAAIVDMVPGKPMCVESFSDYPPLG

SEQ ID NO: 18 - protein, 62 aa, Homo sapiens [sequence omitted]

SEQ ID NO: 19 - protein, 59 aa, Homo sapiens
MVPGKPMCVESFSDYPPLGRFAVRDMRQTVAVGVIKAVDKKAAGAGKVTKSAQKAQKAK

SEQ ID NO: 20 - protein, 88 aa, Homo sapiens [sequence omitted]

SEQ ID NO: 21 - protein, 455 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 22 - protein, 415 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 23 - protein, 222 aa, Homo sapiens
MGKEKTHINIVVIGHVDSGKSTTTGHLIYKCGGIDKRTIEKFEKELLRWERAPSSMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYALYWIATRLTLHASLLS

SEQ ID NO: 24 - protein, 190 aa, Homo sapiens [sequence omitted]

SEQ ID NO: 25 - protein, 459 aa, Homo sapiens [sequence omitted]

SEQ ID NO: 26 - protein, 296 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTVRMAMPVEPRCLRLWTASYHQLVQLTSPCACLSRMSTKLVVLVLFLLAEWRLVFSNPVWWSPLLQSTLQCGLQCQECVCQGCSSWQRCW

SEQ ID NO: 27 - protein, 205 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 28 - protein, 208 aa, Homo sapiens [sequence omitted]

SEQ ID NO: 29 - protein, 375 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAEL

SEQ ID NO: 30 - protein, 140 aa, Homo sapiens [sequence omitted]

SEQ ID NO: 31 - protein, 29 aa, Homo sapiens [sequence omitted]

SEQ ID NO: 32 - protein, 108 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAQDTETLSKHDYRDISG

SEQ ID NO: 33 - protein, 87 aa, Homo sapiens
MGKEKTHINIVVIGHVDSGKSTTTGHLIYKCGGIDKRTIEKFEKEAAEMGKGSFKYAWVLDKLKTRYGGHLCSSQRYNGSKICRNAP

SEQ ID NO: 34 - protein, 86 aa, Homo sapiens [sequence omitted]

SEQ ID NO: 35 - protein, 83 aa, Homo sapiens [sequence omitted]

SEQ ID NO: 36 - protein, 218 aa, Homo sapiens
MGKGSFKYAWVLDKLKAERERGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSARKLRRLNEYYP

SEQ ID NO: 37 - protein, 149 aa, Homo sapiens
MITGTSQADCAVLIVAAGVGEFEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 38 - protein, 75 aa, Homo sapiens
MLEPSANMPWFKGWKVTVRMAMPVEPRCLRLWTASYHQLVQLTSLAPASPGCLQNLVFSNPVWWSPLLQSTLQRK

SEQ ID NO: 39 - protein, 108 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALQVTLRRLF

SEQ ID NO: 40 - protein, 113 aa, Homo sapiens [sequence omitted]

SEQ ID NO: 41 - protein, 127 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTKKSETLTGSTERN

SEQ ID NO: 42 - protein, 131 aa, Homo sapiens [sequence omitted]

SEQ ID NO: 43 - protein, 183 aa, Homo sapiens [sequence omitted]

SEQ ID NO: 44 - protein, 92 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKVS

SEQ ID NO: 45 - protein, 297 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKVCTILLICPA

SEQ ID NO: 46 - protein, 254 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRGNNKFLFLESVYGRCNFLF

SEQ ID NO: 47 - protein, 212 aa, Homo sapiens
MAMVSEFLKQAWFIENEEQEYVQTVKSSKGGPGSAVSPYPTFNPSSDVAALHKAIMVKGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 48 - protein, 306 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 49 - protein, 269 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIGM

SEQ ID NO: 50 - protein, 284 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 51 - protein, 282 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 52 - protein, 220 aa, Homo sapiens [sequence omitted]

SEQ ID NO: 53 - protein, 99 aa, Homo sapiens
MNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 54 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 55 - protein, 61 aa, Homo sapiens
LHKAIMVKGVDEATIIDILTKRNNAQRQQIKAAYLQETGKPLDETLKKALTGHLEEVVLAL

SEQ ID NO: 56 - protein, 61 aa, Homo sapiens
LRAAMKGLGTDEDTLIEILASRTNKEIRDINRVYREELKRDLAKDITSDTSGDFRNALLSL

SEQ ID NO: 57 - protein, 61 aa, Homo sapiens
YEAGERRKGTDVNVFNTILTTRSYPQLRRVFQKYTKYSKHDMNKVLDLELKGDIEKCLTAI

SEQ ID NO: 58 - protein, 61 aa, Homo sapiens
LHQAMKGVGTRHKALIRIMVSRSEIDMNDIKAFYQKMYGISLCQAILDETKGDYEKILVAL

SEQ ID NO: 59 - protein, 294 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 60 - protein, 171 aa, Homo sapiens
MKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKVCTILLICPA

SEQ ID NO: 61 - protein, 140 aa, Homo sapiens [sequence omitted]

SEQ ID NO: 62 - protein, 95 aa, Homo sapiens [sequence omitted]

SEQ ID NO: 63 - protein, 87 aa, Homo sapiens [sequence omitted]

SEQ ID NO: 64 - protein, 70 aa, Homo sapiens [sequence omitted]

SEQ ID NO: 65 - protein, 54 aa, Homo sapiens
MAMVSEFLKQAWFIENEEQEYVQTVKSSKGGPGSAVSPILPSIHPRMSLPCIRP

SEQ ID NO: 66 - protein, 253 aa, Homo sapiens
MHSWERLAVLVLLGAAACAAPPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 67 - protein, 214 aa, Homo sapiens
MVSLQLRGGHFCGATLIAPNFVMSAAHCVANVADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 68 - protein, 195 aa, Homo sapiens
MVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPMPLPRWHSL

SEQ ID NO: 69 - protein, 143 aa, Homo sapiens
MGKLRPGEGRVIITAPCDALTICPHRHSSTGRPPSTPTCRCPAAGSGTPPGQRVQCLAMGWGLLGRNRGIASVLQELNVTVVTSLCRRSNVCTLVRGRQAGVCFGDSGSPLVCNGLIHGIASFVREAAPQGSTPMPLPRWHSL

SEQ ID NO: 70 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 71 - protein, 342 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFVRALGVPLLPTRSQPGLQQQAPWLDPRRDFPTLTGGGQVGRASQSSFPTLSASTGGLRQPLGLQRANPRNCLLRPGRLRLRALPRCLCPGGTVCKLDRLYHPTLRGQPLSPPPGPGPGQQDPLRRAARVTSAAHTHTLQHLAQ

SEQ ID NO: 72 - protein, 176 aa, Homo sapiens
MGTRLAGARWAQRVGHHQRQRAGGPAAGSGTPPGQRGAVPGHGLGPSGQEPWDRQRPAGAQRDGGDVPLPSQQRLHSREGPAGRRLFRGLRQPLGLQRANPRNCLLRPGRLRLRALPRCLCPGGTVCKLDRLYHPTLRGQPLSPPPGPGPGQQDPLRRAARVTSAAHTHTLQHLAQ

SEQ ID NO: 73 - protein, 114 aa, Homo sapiens [sequence omitted]

SEQ ID NO: 74 - protein, 257 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQGRREGAGSPGRALPVAAGALQAPVRRAPRSAPPGQPARHHRPRPPAATAVGEGHTGPCCAPPALAARGPRRGTGNSLRRGRLGHSQPRGPPPGQPAARALASAGPRHLQPAHAPRRRHHRALDVRGEQSPGQLQG

SEQ ID NO: 75 - protein, 209 aa, Homo sapiens
MTLGRRLAVFSSPVSCRPCCWGHRAGLGDCGGPASAAPRVALHGVPAAARRPLLRRHPDCAQLRHVGRALRGECGRREGAGSPGRALPVAAGALQAPVRRAPRSAPPGQPARHHRPRPPAATAVGEGHTGPCCAPPALAARGPRRGTGNSLRRGRLGHSQPRGPPPGQPAARALASAGPRHLQPAHAPRRRHHRALDVRGEQSPGQLQG

SEQ ID NO: 76 - protein, 75 aa, Homo sapiens
MAVMAPPNPRPATLGALALTQTWAGSHSMRYFFTSVSRPGRGSPASSQWATWTTRSSCGSTATPRARGWSRGRRG

SEQ ID NO: 77 - protein, 75 aa, Homo sapiens
MAVMAPRTLVLLLSGALALTQTWAGSHSMRYFFTSVSRPGRGSPASSQWATWTTRSSCGSTATPRARGWSRGRRG

SEQ ID NO: 78 - protein, 179 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAMWRSS

SEQ ID NO: 79 - protein, 362 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSSPIIFSCSREVGLRCLHLCLNFMVH

SEQ ID NO: 80 - protein, 365 aa, Homo sapiens [sequence omitted]

SEQ ID NO: 81 - protein, 194 aa, Homo sapiens
MRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSHSL

SEQ ID NO: 82 - protein, 297 aa, Homo sapiens
MEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 83 - protein, 153 aa, Homo sapiens
MTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 84 - protein, 300 aa, Homo sapiens
MAVMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 85 - protein, 396 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSLYSVRQLPCVGLRGKSCSCPSLCDLKNPDFVSAKAPACVCVRVGIM

SEQ ID NO: 86 - protein, 306 aa, Homo sapiens
MEPRAPWIEQEGPEYWDGETRKVKAHSQTHRVDLGTLRGYYNQSEAASHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGKSCSCPSLCDLKNPDFVSAKAPACVCVRVGIM

SEQ ID NO: 87 - protein, 348 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSPIIFPVPERWG

SEQ ID NO: 88 - protein, 339 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDR

SEQ ID NO: 89 - protein, 324 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPHRGHHCWPGSLWSCDHWSCGRCCDVEEEELR

SEQ ID NO: 90 - protein, 252 aa, Homo sapiens
MEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVASLLAWFSLEL

SEQ ID NO: 91 - protein, 281 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDRSRDTPAMCSMRVCPSPSP

SEQ ID NO: 92 - protein, 187 aa, Homo sapiens
MEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELR

SEQ ID NO: 93 - protein, 212 aa, Homo sapiens
MAVMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQIEKEGATLRLQAVAVPRALMCLSQLVKCETAALCGTERQELFLPFPL

SEQ ID NO: 94 - protein, 96 aa, Homo sapiens
MRVMAPRTLLLLLSGALALTETWACSHSMRYFDTACPGPAAESPTSSQWATWTTRSSCGSTATPRVQEGSRGAVGGAGGAGVLGPGDTEVQAPGTD

SEQ ID NO: 95 - protein, 110 aa, Homo sapiens
MRVMAPRTLLLLLSGALALTETWACSHSMRYFYTAVSRPSRGEPHFIAVGYVDDTQFVRFDSDAASPRGSRGRRGWSRRGRSIGTGRHRSTSASSAGMTSPPTTARITSP

SEQ ID NO: 96 - protein, 291 aa, Homo sapiens
MRVMAPRTLLLLLSGALALTETWACSHSMRYFYTAVSRPSRGEHHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQRGSVGASGESRIHVPCAARGRRALT

SEQ ID NO: 97 - protein, 293 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGCPGCPSCPRSCGGCCDV

SEQ ID NO: 98 - protein, 245 aa, Homo sapiens
MYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 99 - protein, 366 aa, Homo sapiens [sequence omitted]

SEQ ID NO: 100 - protein, 366 aa, Homo sapiens
MRVMAPRTLILLLSGALALTETWACSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 101 - protein, 327 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 102 - protein, 352 aa, Homo sapiens
MRVMAPRTLACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 103 - protein, 338 aa, Homo sapiens
MRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 104 - protein, 326 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWGGKGGSCSQAASSNSAQGSDESLIACKA

SEQ ID NO: 105 - protein, 350 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGELLSVWD

SEQ ID NO: 106 - protein, 228 aa, Homo sapiens
MYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGFLHTSPL

SEQ ID NO: 107 - protein, 343 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSPPPCPP

SEQ ID NO: 108 - protein, 343 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPDHLSCSREVGLDVSISVSNSWCTELQLLTSLMKLRT

SEQ ID NO: 109 - protein, 174 aa, Homo sapiens
MSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGCKSPSP

SEQ ID NO: 110 - protein, 308 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIAPNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWKSLRQLPVWD

SEQ ID NO: 111 - protein, 181 aa, Homo sapiens
MYGCDLGPDGRLLRGYDQSAYDGKDYIALNEDLRSWTAADTAAQITQRKWEAARRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSSQPTIPIVASLLAWLSWLS

SEQ ID NO: 112 - protein, 83 aa, Homo sapiens
MRVMAPRTLLLLGRRALALTETQKYKRQAQTDRVNLRKLRGYYNQSEAGSHTLQRMYGCDLGPTGASSAGMTSPPTTARITSP

SEQ ID NO: 113 - protein, 154 aa, Homo sapiens
MRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRA

SEQ ID NO: 114 - protein, 169 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRLRRQGLHRPERGPALLDRRGHSGSDHPAQVGGGP

SEQ ID NO: 115 - protein, 313 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKIHVPYAARGAARAPHPELGAIFPAHHPHHGHRCWPGCPGCPSCPWSCGHRYDVVTATVPRALMSLSSLVKPETAACVGLRCRISSHLSFVTSRASGISFCKGT

SEQ ID NO: 116 - protein, 258 aa, Homo sapiens
MRVMAPRTLLLLLSGALALTETWACSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELWWLL

SEQ ID NO: 117 - protein, 28 aa, Homo sapiens
VIILNHPGQISAGYAPVLDCHTAHIACK

SEQ ID NO: 118 - protein, 14 aa, Homo sapiens
FIAVGYVDDTQFVR

SEQ ID NO: 119 - protein, 13 aa, Homo sapiens
AYLEGETCVEWLR

SEQ ID NO: 120 - protein, 24 aa, Homo sapiens
DGEDQTQDTELVETRPAGDGTFQK

SEQ ID NO: 121 - protein, 13 aa, Homo sapiens
WAAVVVPSGQEQR

SEQ ID NO: 122 - protein, 6 aa, synthetic construct
  REGION 1..6: note = Description of Artificial Sequence: Synthetic 6xHis tag
HHHHHH